(19) United States
(12) Patent Application Publication
(10) Pub. No.: US 2010/0003292 A1
(43) Pub. Date: Jan. 7, 2010

(54) FINE-GRANULOMETRY FUNGAL EXTRACT CHITINE-GLUCANE

(76) Inventors: Sandrine Gautier, Liege (BE); Jean-Michel Bruyere, Liege (BE); Véronique Maquet, Berloz (BE)

Correspondence Address: CLARK & BRODY, 1090 VERMONT AVENUE, NW, SUITE 250, WASHINGTON, DC, 20005, US

(21) Appl. No.: 12/312,656

(22) PCT Filed: Nov. 20, 2007
(86) PCT No.: PCT/EP2007/062601

§ 371 Date: Sep. 4, 2009

(30) Foreign Application Priority Data
Nov. 20, 2006 (FR) 0654998

(51) Int. Cl. A61K 8/02; A23L 1/28

(52) U.S. Cl. 424/401; 514/55; 426/655

(57) ABSTRACT

The present invention relates to a chitin-glucan copolymer in the form of micrometric particles.

The present invention in particular provides a composition containing a chitin-glucan copolymer in the form of micrometric particles for the preparation of cosmetic compositions, and in particular dermatological or dermocosmetic compositions.

In particular, the present invention relates to a cosmetic composition for face or body care, such as hydrating, firming or smoothing the skin, and exercising an anti-aging effect, including an anti-wrinkle effect.

The purpose of the invention is also to provide a porous material that can for example be used in tissue engineering or cell culture, or that can be used as a material in the cosmetics or pharmaceutical industry.

The invention relates to a chitin-glucan copolymer in the form of a powder with a fine and controlled particle size, in particular with a very fine particle size, that can be used especially in the cosmetics field, and particularly to the use of a chitin/beta-glucan copolymer for preventing or reducing the signs of skin aging.

The invention also relates to such a polymer in the form of porous materials, in particular for its use in tissue engineering.

### PRIOR ART

It is known that certain polysaccharides exert a hydrating action with respect to the upper layers of the epidermis, or even make it possible to prevent or reduce certain signs of skin aging. In particular, ingredients based on beta-glucans, schizophyllan, xyloglucan, hyaluronic acid, galactomannans and chitin are commonly used in cosmetic care products.

These polysaccharides differ from ingredients of the alpha-hydroxy acid type, which act by desquamating the epidermis, which promotes rapid cell renewal but can impair the lower layers of the epidermis. Most anti-aging ingredients act on the renewal of skin cells or collagen. Now, a mechanism for the renewal of collagen poses the problem of compatibility with the legislation in terms of cosmetic products, for which transcutaneous penetration cannot be claimed. Treating the signs of skin aging goes beyond the notion of wrinkles and cell renewal, and it is rare today to find available active agents that address the problem in an overall and really effective manner.

Among polysaccharides with an “anti-aging” effect, beta-glucans, derived from yeasts, from fungi, from cereals or from plants, are a family of pure polysaccharides consisting of D-glucose units linked to one another by beta-type linkages, the carbons that are linked varying according to the species from which they are extracted, with a more or less branched structure: beta(1,3)(1,6) for beta-glucans derived from the yeast Saccharomyces cerevisiae; beta(1,3) for the main chain (branched in the beta(1,6)-position with short chains) of schizophyllan, derived from the fungus Schizophyllum commune, beta(1,4) for beta-glucans derived from cereals such as oats, barley or wheat; beta(1,4) for the main chain (branched in the beta(1,6)-position with short chains) for xyloglucan derived from plants. The solubility of beta-glucans in an aqueous medium depends on their structure, on the length of the macromolecular chains and on the three-dimensional organization of the chains.

Beta-glucans are looked upon favorably in the cosmetics industry for their revitalizing and anti-inflammatory effects, protective effects against UV radiation, soothing, immunostimulant, anti-aging, anti-wrinkle and anti-acne effects, etc. (which effects differ according to the molecule considered), which result in an improvement in the symptoms of skin aging or of acne. The desired beta-glucans in cosmetics are generally water-soluble so that they can be incorporated into the aqueous phase of emulsions, which limits the choice in terms of molecules that can be used in cosmetics. In fact, insoluble beta-glucans have very advantageous cosmetic and dermatological properties, but cannot be incorporated into cosmetic formulas because they are in the form of particles that are hard, irritant, etc. The few water-soluble beta-glucans that can be used in cosmetics are provided in the form of beta-glucan-rich extracts or solutions.

Furthermore, cosmetic uses of water-soluble derivatives of chitin (mainly carboxymethylchitin and chitosan) are known, for example in care creams, as a film-forming agent, moisturizer, agent for improving the appearance of skin subjected to cellulite, for example, or also for the preparation of microspheres. However, chitin for cosmetic use, and derivatives thereof, are industrially obtained from shells of shellfish—shrimp, crab—shellfish being one of the main agents responsible for allergies. Cases of allergies to creams containing chitin derivatives have, moreover, been published (Cleenewerck M B, Martin P, Laurent D. Allergic contact dermatitis due to a moisturizing body cream with chitin. Contact Dermatitis 31, 196, 1994; Pereira F, Pereira C, Lacerda M H. Contact dermatitis due to a cream containing chitin and a carbitol. Contact Dermatitis 38, 290, 1998).

To the inventors' knowledge, chitin (the polymer consisting of N-acetyl-D-glucosamine units) obtained starting from the shells of shellfish or starting from microscopic algae is not known as being able to prevent or reduce the effects of skin aging. Chitosan, which is the polymer derived from chitin, carrying cationic charges, consisting of D-glucosamine/N-acetyl-D-glucosamine units, its derivatives (succinamide) and its salts (for example, lactate, ascorbate, glycolate, succinate), are used in the cosmetics industry for their substantive, film-forming, hydrating, antimicrobial and anti-aging properties, properties for improving the appearance of cellulite, and properties for improving the feel of formulas.

The inventors have already shown that it is possible to isolate and purify a copolymer composed of two types of chains, chitin [poly(N-acetyl-D-glucosamine)] and beta-glucan [poly(D-glucose)], from fungi. The walls of the cells of the mycelium of certain fungi, such as Aspergillus niger (of the Ascomycete type), consist of the two polysaccharides covalently bonded to one another in a three-dimensional network, called “chitin-glucan”.

Now, this chitin-glucan copolymer can be advantageously produced, in a highly pure and profitable manner, by means of a process of successive steps, as described in patent EP1483299B1 (US 2005/130273 A1 or WO 03/068824 A1). A fine, white, unscented powder is obtained, the chitin-glucan copolymer content of which is greater than 90%. This powder is not soluble in any solvent, neither aqueous nor organic, which a priori compromises its use in the cosmetics industry. Various applications are described in patent applications FR 05 07066 and FR 06 51415.

Medical uses of compositions based on fungal extracts containing chitin and beta-glucans have previously been described, in particular as wound-healing active agents. However, these various uses, for example in the form of dressings, are not a priori suitable for a cosmetic formulation.

### OBJECTIVES OF THE INVENTION

The main objective of the invention is to solve the new technical problem consisting of the provision of a chitin-glucan copolymer in a form suitable for cosmetic use, and in particular in dermocosmetics or in dermatology, and/or suitable for medical or pharmaceutical use, and/or suitable for use as a food supplement for humans or animals.

An objective of the invention is also to solve the new technical problem consisting of the provision of a chitin-glucan copolymer in the form of a suspension, of an emulsion or of a dispersion, especially that can be used in the cosmetics field, and in particular in the dermocosmetics or dermatology field.

An objective of the present invention is in particular to provide a dermocosmetic composition for a body and/or face care, such as a hydrating, firming, protecting, anti-wrinkle (described in particular through an evaluation of the contours of the skin by profilometry) or anti-aging care.

An objective of the present invention is also to solve the technical problems mentioned above by providing a substance of natural origin which exhibits innocuousness, skin and ocular tolerance, and very good hypoallergenicity, while at the same time being readily available in large volume, and at a cost compatible with use as a cosmetic ingredient.

An objective of the present invention is also to optimize a food supplement composition that enables easy oral administration and that promotes the bioavailability and the effects on the health of the chitin-glucan copolymer.

An objective of the present invention is also to provide a chitin-glucan powder that makes it possible to adjust and optimize its physicochemical and biological properties according to the use envisaged, such as a dermocosmetic and dermatological composition, a food supplement composition, a functional food composition, a technological aid for the treatment of beverages, or a composition for medical devices, for instance healing products.

An objective of the invention is also to provide a natural substance of non-animal origin and of excellent purity, which is well characterized and obtained by means of a production process which guarantees reproducibility and traceability.

An objective of the present invention is also to provide a natural substance, of polysaccharide type, that is stable as a powder and in suspension, that is easy to formulate, that is compatible with all the ingredients most commonly used, and that allows the preparation of stable cosmetic formulations, the characteristics of which are perfectly suitable for their use, for example with a perfectly homogeneous textile, and the sensory qualities (viscosity, texture, feel) of which are excellent.

An objective of the invention is to provide a cosmetic active agent for preventing or reducing the effects of skin aging, hydrating the skin in a lasting manner, giving it tonicity and/or firming it, giving it a homogeneous and smooth appearance, decreasing the squamous state, protecting it against outside attacks such as dryness and/or heavy metal pollution, and allowing it to restore its barrier function. An objective of the invention is also to provide a cosmetic ingredient that has a capacity to retain water and a considerable viscosity-modifying capacity. An objective of the invention is also to provide a porous material that can be used, for example, in tissue engineering or cell culture or that can be used as a material in the cosmetics or pharmaceutical industry.

### SUMMARY OF THE INVENTION

In order to solve the technical problems mentioned above, the inventors started from the action of the beta-glucan-type substances, in the cosmetics industry, of which they had knowledge. However, it would not, a priori, have been possible for any composition based on a chitin-glucan copolymer to have been useable insofar as the powder obtained, for instance according to the process described in PCT patent application WO 03068824, is not soluble in the aqueous or organic phase.

The inventors have, however, discovered, surprisingly, that it is possible to solve the technical problems mentioned above by using a powder, with a very fine and controlled particle size, of a chitin-glucan copolymer. This solution is entirely surprising since those skilled in the art expected the finely milled particles of this copolymer to also be insoluble and that said copolymer would be in the same form as the insoluble beta-glucans, i.e. in the form of particles that are hard and irritant to the skin.

The inventors have, surprisingly, been able, firstly, to prepare stable dispersions of chitin-glucan, and stable suspensions of chitin-glucan, in particular in water with no additive, and secondly, to prepare stable emulsions containing, in particular, high concentrations of chitin-glucan.

By the term “chitin-glucan”, the inventors mean a chitin-glucan copolymer according to the present invention.

In particular, the invention relates to a polysaccharide of fungal origin comprising predominantly a chitin-glucan copolymer, said polysaccharide having a fine particle size.

The invention also relates to a finely milled powder of a fungal extract comprising at least one finely milled chitin-glucan copolymer.

Advantageously, the particles of fine particle size are made up of at least 70% by weight of particles with a size less than 500 microns (μm), and preferably less than 355 microns (μm).

Again preferably, at least 50%, preferably 60%, by weight of the particles have a size of less than 250 microns (μm), and preferably less than 150 microns (μm).

The expression “particle size of less than X microns” is intended to mean particles which have a size that allows them to pass through a screen whose mesh size is X microns.

One embodiment makes it possible to obtain at least 50% by weight of particles with a size of less than 65 mesh (approximately 149 μm), and preferably less than 100 mesh (approximately 230 μm).

The particle size is advantageously controlled by choosing, in particular after screening or classification, a fraction of specific size according to needs. The fractions to which reference is made in the examples are included herein by way of reference in terms of their generality, in particular with respect to the type of copolymer, which may be any one of those described in the present invention.

Advantageously, the chitin-glucan copolymer comprises a ratio of N-acetyl-D-glucosamine units of the chitin to D-glucose units of the beta-glucans of between 95:5 and 0.15:85 (m/m).

Advantageously, the polysaccharide of fungal origin comprises more than 70% of chitin-glucan copolymer by mass relative to the total mass of the extract of fungal origin, preferably greater than 85%.

Advantageously, the linkages between the D-glucose units are predominantly of beta(1,3) type.

Preferably, the fungal extract is derived from the mycelium of a fungus of the Ascomycete type, and in particular of Aspergillus niger, and/or of a Basidiomycete fungus, and in particular Lentinula edodes (shiitake) and/or Agaricus bisporus.

Advantageously, at least 85% of the chitin part of the chitin-glucan copolymer is N-acetyl-D-glucosamine units, and at most 15% is D-glucosamine units.

The fungal extract of micrometric particle size is preferably a hydrolysate of the chitin-glucan copolymer.

Advantageously, the ratio of chitin to beta-glucan is between 90:10 and 30:70 (m/m).

The invention also relates to a composition comprising a polysaccharide or a fungal extract with a fine particle size as defined above, in particular in the form of a suspension, of an emulsion or of a dispersion.

Advantageously, the composition is a cosmetic composition, in particular a dermocosmetic or dermatological composition.

Preferably, the polysaccharide or the fungal extract with a fine particle size is used at a concentration of between 0.01% and 10%, and preferably between 0.05% and 5%, by weight of the total composition.

The invention also relates to the use of a composition as defined above, for practicing cosmetic care, preferably dermocosmetic or dermatological care, characterized in that the care is chosen from the group consisting of body or face care, for improving, in particular in a lasting and significant manner, the hydration of the skin, increasing the capacity of the skin to retain water, in particular in the long term, improving the barrier function of the skin, exerting an anti-aging effect, improving the appearance of the skin, improving the homogeneity of the skin, especially by making it smoother, more homogeneous, softer, healthier, improving the firmness and the tonicity of the skin, and promoting attachment of the epidermis to the dermis.

The expression “product having an anti-aging effect” is intended to mean a product or a composition which makes it possible to slow down skin aging, in particular through improving the protection of the skin and/or the defense activities of the skin, reducing the effects of outside attacks, such as radiation, dryness of the air, cold, pollution, especially by heavy metals, or attacks releasing free radicals, and especially attacks by UV radiation, and also through reducing skin wrinkles.

The invention also relates to a composition as defined above, for decreasing the depth of wrinkles, or slowing down or preventing the appearance of wrinkles.

The cosmetic compositions advantageously comprise from 0.1% to 2% of the chitin-glucan copolymer with a fine particle size.

The invention also relates to the use of a composition as defined above, as a food supplement composition, preferably for obtaining an effect chosen from the group consisting of an antioxidant, blood-cholesterol-lowering or blood-lipid-lowering effect, a stimulatory effect on the immune system, a hypoglycemic effect, in particular in the case of diabetes, and an effect consisting in preventing and/or treating and/or combating a pathology chosen from the group consisting of dyslipidemia, atherosclerosis, obesity, an obesity-related disease, a cardiovascular disease, metabolic syndrome, diabetes and hyperuricemia.

Preferably, in this food supplement composition, the fungal extract copolymer with a fine particle size is used as an active ingredient.

The invention also relates to a pharmaceutical composition comprising, as active ingredient, at least one copolymer or one extract of fungal origin, as defined above.

The invention relates in particular to the use, in tissue engineering, of a porous material obtained from the polysaccharide or extract of fungal origin according to the present invention, and therefore also relates to the porous material obtained from the polysaccharide or extract of fungal origin according to the present invention. This porous material may be obtained in particular by lyophilization.

The invention also relates to the use of at least one polysaccharide or one extract of fungal origin, as defined above, as an excipient of a composition, in particular a cosmetic composition, preferably a dermatological or dermocosmetic composition.

The invention also relates to a process for preparing a fungal extract with a fine particle size, comprising:

a) the extraction and purification of a chitin-glucan copolymer from a fungal biomass, said copolymer of this fungal extract being insoluble in water or an organic solvent,
b) steps, which are simultaneous or separate independently of their order, of filtration, drying, milling and classification of the particles, starting from the dry or solvated chitin-glucan, making it possible to obtain micrometric particles of which at least 70% by weight, preferably 75%, and more preferably 80%, of the particles have a size of less than 500 microns (μm), and preferably less than 355 microns (μm).

Preferably, step b) of the process for preparing the fungal extract with a fine particle size makes it possible to obtain at least 50%, preferably 60%, and more preferably 70%, by weight of the total particles obtained, having a size of less than 250 microns, preferably less than 125 μm.

It is thus possible, in step b), to carry out a simultaneous filtration, drying and milling step, and then a separate classification step.

The invention also relates to the device or equipment for carrying out the process according to the present invention.

### DETAILED DESCRIPTION OF THE INVENTION

The compositions according to the invention make it possible to obtain formulations with a pleasant and soft feel, which is very advantageous in the cosmetics field especially.

In particular, the stability, the texture, the color, the feel, the viscosity and the rheology of the emulsions or suspensions obtained are perfectly suitable for the production of face or body care creams, including for babies. Finally, the excellent skin tolerance (in vitro and in vivo in humans) and ocular tolerance (in vitro) and also the hypoallergenicity (in vivo in humans, according to the Maibach-Marzulli procedure) of a chitin-glucan copolymer have been established.

The solution proposed by the inventors is all the more advantageous since a purified chitin-glucan copolymer derived from fungal sources, in particular but not exclusively of Ascomycete type, is available in large amounts as an industrial by-product.

It is in particular preferable to use the mycelium of Aspergillus niger as fungal source.

According to the invention, the chitin-glucan copolymer extracted from the Ascomycete-type fungal mycelium can be readily formulated, although it is water-insoluble, in particular in the form of a cosmetic composition.

The inventors have discovered, surprisingly, that, when the chitin-glucan compound derived from fungal sources is in the form of a powder with a fine particle size, it is entirely suitable for the preparation of a composition that makes it possible to solve the technical problems mentioned above.

The chitin-glucan powder advantageously obtained according to the process described in PCT patent application WO 03/068824, or in French patent application FR 0507066, is prepared in such a way as to obtain a fine and controlled particle size, in particular by means of filtration, milling, drying and/or particle classification processes. When a fine particle size is desired, a process for obtaining particles in which at least 70% by weight, preferably 75%, and more preferably 80%, of the particles have a size of less than 500 microns (μm), and preferably less than 355 microns (μm), is selected.

Advantageously, the particles are made up of at least 50% by weight, preferably 60%, and more preferably 70%, of particles with a size of less than 250 microns (μm), and preferably less than 150 microns (μm).

Preferably, a process for preparing the chitin-glucan powder is carried out in such a way as to obtain predominantly particles with a size of less than 250 microns.

Advantageously, the particles according to the present invention consist essentially of particles with a size of less than 125 μm, or even less than 90 μm, and in particular are obtained after classification so as to obtain a narrow distribution.

It is possible to carry out the process for preparing the powder by means of any one of the techniques known to those skilled in the art. It is advantageous to select a technique that makes it possible to control the particle size as well as possible and in a well-defined manner.

The term “particle size” is intended to mean the more or less spherical shape, the size and the size distribution of the particles of the chitin-glucan powder. This parameter, which characterizes the powdered ingredient, influences, firstly, the way in which it can be formulated, i.e. in which it can be incorporated into a solid or liquid composition such as a food matrix, a cosmetic cream, a dietary or cosmetic liquid, or a medical or pharmaceutical device. According to the particle size obtained, and in particular according to the appearance or the final shape desired, the composition obtained will be more or less homogeneous.

The particle size and the size distribution of the particles are characterized by conventional techniques such as light diffraction (for example, a Mastersizer 2000 laser diffraction system from Malvern Instruments), scanning electron microscopy followed by image analysis, or screening on successive screens followed by gravimetric measurement.

Advantageously, it has been established, surprisingly, that, by adjusting the size distribution of the particles so as to obtain a particle size of less than 125 μm, it is possible to obtain a homogeneous cream containing the chitin-glucan polymer. This makes it possible in particular to incorporate this copolymer after formation of an emulsion, even at a high concentration (for example 2%), and to achieve a completely homogeneous texture, the sensory qualities of which (viscosity, texture, feel) are excellent. The fraction of the particles having the smallest size is therefore advantageously used.

When the size of the particles is larger, the ingredient no longer provides these sensory qualities, the product forms grains that are palpable when the cream is spread, and/or the formulation is not stable over time, which is not desired in the case of a topical composition, and in particular in a care cream.

Advantageously, a powder with a fine and controlled particle size can be used for the preparation of “functional” food products, such as biscuits, pastes, confectionery products, dietetic bars, breads, drinks, butters, margarines, etc.

Advantageously, the powder with a fine particle size can be used in the form of an aqueous dispersion, and can be part of the composition of medical devices such as healing and/or hemostatic systems. Surprisingly, the chitin-glucan with a fine particle size can be used in the form of a cohesive porous material that has good mechanical stability and a porosity of greater than 80%, preferably greater than 90%, by means of porogenic techniques well known to those skilled in the art. The porous material can be prepared either starting from a concentrated aqueous dispersion in the form of a paste of chitin-glucan alone, or starting from a dispersion of a mixture of chitin-glucan and other insoluble and dispersible compounds, or starting from a dispersion of chitin-glucan in an aqueous phase in which a polymer or another substance is solubilized. The porosimetry and the mechanical properties of the materials obtained depend on the formulation parameters, in particular on the particle size of the chitin-glucan, on the composition of the mixture, on the concentration of the starting dispersion, and also on the dispersion implementation parameters.

For the preparation of dermocosmetic and dermatological products, the chitin-glucan with a fine particle size, with particles having a size that is preferably less than 125 μm, advantageously has good affinity both with components present in the aqueous phase and in the oily phase, which promotes the incorporation process.

The chitin-glucan powder with a fine particle size can be produced industrially according to various processes, depending on the intended particle size, either starting from chitin-glucan in the form of a dry powder, or starting from chitin-glucan solvated in an aqueous or organic medium, or else starting from chitin-glucan incorporated into a more complex medium such as an oil-in-water or water-in-oil emulsion. The inventors mean, by “processes for preparing a powder with a fine particle size”, any of the solid-liquid and solid-solid processes of filtration, drying, milling, homogenization, particle size reduction and particle classification applied to solids, to solvated solids and to solvated complex media such as emulsions, colloidal suspensions, etc.

The various industrial separation processes can be used while starting from solvated chitin-glucan, for instance with a conical drier, a Nütsche filter, a plate filter, a band filter, a fluidized bed drier, and spraying equipment, so as to achieve complete or partial drying of the chitin-glucan. The processes can be applied to the solvated chitin-glucan as it is, or after milling of the solvated chitin-glucan. The various industrial fragmentation processes for obtaining powders with a fine and controlled particle size can be applied to the completely or partially dried product, or to the solvated product, for instance flail, hammer, roller, knife, blade, disc and counter-airjet milling processes, and disintegrating processes, for example ultrasonic and micronization processes. The various industrial processes for separating powders can be used in order to decrease the breadth of the size distribution or to select a specific size, for example with dynamic and static screening and classification equipment.

Thus, the present invention relates to a process for obtaining a chitin-glucan powder of fungal origin that is water-insoluble or insoluble in an organic solvent, and that has a fine particle size, for preparing particles of the chitin-glucan copolymer that are stable in an aqueous or organic solution, in particular for preparing a suspension or emulsion.

The step for obtaining the powder with a fine particle size is carried out before or after the preparation of the emulsion, of the suspension or of the dispersion.

This suspension or dispersion or emulsion contains substances generally used in the cosmetics field and advantageously allows the formulation of a cosmetic composition.

Cosmetic compositions generally contain from 0.01% to 10% by weight of the compositions according to the present invention, in particular from 0.01% to 10% by weight in the form of a suspension or emulsion, relative to the weight of the total composition.

The inventors mean, by “derivatives of the chitin-glucan copolymer”, all the compounds that are obtained starting from chitin-glucan, by physical or chemical modification, according to physical, chemical and enzymatic processes.

The present invention relates in particular to a polysaccharide of fungal origin comprising a polymer comprising beta-glucan chains, said beta-glucan chains consisting essentially of linkages of D-glucose units via bonds in the (1,3)-position, and preferably comprising at least 80% by mass of beta-glucan chains in which the D-glucose linkage is in the (1,3)-position relative to the percentage of the total mass of beta-glucan, in particular for the manufacture of a cosmetic formulation.

A chitin-glucan copolymer can be advantageously obtained according to the process described in PCT patent application WO 03/068824, and French patent application FR 0507066 filed by KitoZyme S. A. on 4 Jul. 2005. This process is described in particular in application FR 0507066 on page 18, line 14 et seq. Aspergillus niger is preferably used as fungal source in this process.

The D-glucose-unit linkage and the proportion between the alpha(1,6)-chitin and beta-glucan chains depend on the fungus and on the strain. For example, it has been shown, by the inventors, that an Aspergillus niger mycelium contains the chitin-glucan copolymer with a ratio by mass of chitin to beta-glucan of between 30:70 and 60:40, with a D-glucose-unit linkage mainly of the beta(1,3) type.

The copolymer is generally in the form of a white powder. It is essentially insoluble in aqueous and organic solvents irrespective of the temperature and the pH. It is hygroscopic, being generally capable of absorbing approximately 10 times its mass in water. This chitin-glucan powder can, for example, be produced by means of industrial processes in such a way as to obtain a product with a fine particle size according to the present invention.

The present invention relates to an extract, which is advantageously purified, of a fungal source, and preferably of the mycelium of Ascomycete-type fungi such as Aspergillus niger. The hydrolysates of the purified extracts, i.e. the copolymers of chitin and beta-glucan of lower molecular mass, are also part of the invention. The present invention also covers, under the term “chitin-glucan copolymer” or “chitin-glucan”, all the compounds obtained starting from chitin-glucan, by physical or chemical modification of the copolymer, according to a physical, chemical or enzymatic process, insofar as the properties of the chitin-glucan copolymer remain equivalent for the applications envisaged, and insofar as the copolymers are insoluble in water and an organic solvent within the particle size range of the present invention, but can be formulated in the form of a dispersion, of an emulsion or of a suspension.

The availability and the quality in particular of Aspergillus niger, which is a co-product of the industrial production of citric acid for the food and pharmaceutical industry, make it a starting material of choice for uses in the cosmetics industry. Other fungal sources containing the chitin and beta-glucan polysaccharides can also be used, for instance Basidiomycetes, in particular the fungi Lentinula edodes (shiitake) and Agaricus bisporus.

The inventors mean, by “polysaccharides of fungal origin”, the purified extracts of fungal cell walls composed predominantly of chitin and beta-glucan polysaccharides, in the form of copolymers, and derivatives thereof. The purified extracts preferably comprise a chitin-glucan content of greater than 70% by mass relative to the total mass of the extract, preferably greater than 80%, preferably greater than 85% and more preferably greater than 90%.

The inventors mean, by “chitin-glucan”, a pure copolymer extracted from the cell walls of fungi which consists of links of N-acetyl-D-glucosamine units and, optionally, of a minor proportion of D-glucosamine units linked to one another by (1,6)-type linkages in the alpha conformation (chitin link), and of links of D-glucose units linked to one another by linkages of beta(1,3), beta(1,3)(1,6) or beta(1,3)(1,4) type, and preferably beta(1,3) type (beta-glucan links).

It is generally accepted that fungal cell wall polysaccharides can be separated into two groups according to their solubility in an alkaline medium, and that the cell wall backbone is insoluble. It is also known that the insoluble fraction consists of chitin and of beta-glucan polymers, in variable proportions depending on the species, that the beta-glucan units are linked by linkages of variable structure, and that the bond between the chitin and beta-glucan links is stable, as shown, for example, by Siestma & Wessels for Saccharomyces cerevisiae (Zygomycete), Neurospora crassa (Ascomycete), Aspergillus nidulans (Ascomycete) and Coprinus cinereus (Basidiomycete) [Siestma J H & Wessels J G. (1981) Solubility of (1,3)-beta-D-(1,6)-beta-D-glucan in fungal walls: importance of presumed linkage between glucan and chitin. (1981) J. Gen. Microbiol. 125:209]. It is known that the chitin and beta-glucan links of the insoluble fraction of Aspergillus niger are linked to one another covalently, as mentioned, for example, by Stagg C M and Feather M S [Biochim. Biophys. (1973) Acta 320:64]. Methods for determining the nature of the covalent linkage between the chitin and beta-glucan links have been described, for example, by Fontaine et al., for Aspergillus fumigatus [Fontaine T, Simenel C, Dubreucq G, Adam O, Delepierre M, Lemoine J, Vorgias C E, Diaquin M & Latgé J P. (2000) Molecular organization of the alkali-insoluble fraction of Aspergillus fumigatus cell wall, J. Bio. Chem. 275:27594], and by Kollar et al., for the yeast Saccharomyces cerevisiae [Kollar R, Petrakovas E, Ashwell G, Robbins P & Cabib E. (1995) Architecture of the yeast cell wall, the linkage between chitin and beta(1,3)glucan, J. Biol. Chem. 270:1170].

The fungal extract according to the present invention can be obtained from the mycelium cell wall of fungi of various groups, including the Zygomycete group, the Basidiomycete group, the Ascomycete group (of which Aspergillus niger is part) and the Deuteromycete group, and/or a mixture thereof. Said source of fungi should be chosen so as to allow the extraction of a polysaccharide as defined above and hereinafter. There exists sources of fungi which comprise beta-glucans, but these units are soluble in water in particular, or comprise no or few chains of chitin structure, and therefore do not make it possible to obtain the polysaccharide of the present invention. The present invention covers all fungi that make it possible to obtain the chitin-glucan polymer defined in the present application.

The ratio of the chitin to the beta-glucan is between 95:5 and 5:95, preferably between 70:30 and 10:90 (m/m). The chitin part of the chitin-glucan copolymer is preferably composed of at least 85% of N-acetyl-D-glucosamine units and at most 15% of D-glucosamine units, preferably of at least 90% of N-acetyl-D-glucosamine units and at most 10% of D-glucosamine units.

The invention relates in particular to a suspension or a dispersion comprising a solvent and at least one copolymer with a fine particle size according to the present invention. This suspension or this dispersion is prepared according to the usual methods.

The invention also relates to an emulsion comprising the copolymer with a fine particle size according to the present invention. This emulsion is prepared according to the usual methods, with either water or oil as continuous phase.

Advantageously, the emulsion is first prepared, and then the chitin-glucan copolymer is added. This makes it possible in particular to prepare the emulsion under the temperature conditions usually applied industrially for its preparation, without taking the risk of degrading the copolymer.

The compounds according to the present invention are prepared in particular in the form of cosmetic or pharmaceutical compositions, preferably in topical form. As a result, for these compositions, the excipient contains, for example, at least one compound chosen from the group consisting of preserving agents, antioxidants, stabilizers, conditioners, moisturizers, emollients, emulsifiers, surfactants, thickeners, matting agents, texturing agents, agents for providing sheen, film-forming agents, solubilizing agents, pigments, dyes, fragrances and sunscreens. These excipients are preferably chosen from the group consisting of amino acids and derivatives thereof, polyglycerols, esters, cellulose polymers and derivatives, lanolin derivatives, phospholipids, sucrose-based stabilizers, natural and synthetic waxes, plant oils, triglycerides, unsaponifiable compounds, silicons and derivatives thereof, protein hydrolysates, liposoluble/water-soluble esters, betaines, aminoxides, glycines and parabens.

As oils that can be used in the composition of the invention, mention may be made, for example, of: hydrocarbon-based oils of animal origin, such as perhydrosqualene; hydrocarbon-based oils of plant origin, such as liquid triglycerides of fatty acids containing from 4 to 10 carbon atoms, for instance triglycerides of heptanoic acid or octanoic acid, or alternatively, for example, sunflower oil, maize oil, soybean oil, marrow oil, grapeseed oil, sesame oil, hazelnut oil, apricot oil, macadamia oil, arara oil, castor oil, avocado oil, caprylic/capric acid triglycerides, jojoba oil, shea butter oil; synthetic esters and ethers, in particular of fatty acids, hydroxylated esters, polyol esters; and pentaerythritol esters; linear or branched hydrocarbons of inorganic or synthetic origin, such as volatile or non-volatile paraffin oils, and derivatives thereof, liquid petroleum jelly; fatty alcohols; partially hydrocarbon-based and/or silicone-based fluoro oils; silicone oils such as volatile or non-volatile polymethylsiloxanes (PDMSs) with a linear or cyclic silicone chain, which are liquid or pasty at ambient temperature, in particular cyclopolydimethylsiloxanes (cyclomethicones); phenyl silicones; and mixtures thereof. Various excipients are illustrated in the formulation examples.

The other fatty substances that may be present in the oily phase are, for example, fatty acids containing from 8 to 30 carbon atoms, such as stearic acid, lauric acid, palmitic acid and oleic acid; waxes such as lanolin, beeswax, paraffin waxes, or microcrystalline waxes, synthetic waxes; silicone resins; and silicone elastomers.

Advantageously, the abovementioned compositions are formulated in a form chosen from the group consisting of an aqueous or oily solution, a cream or an aqueous gel or an oily gel, in particular in a pot or in a tube, especially a shower gel, a shampoo; a milk; an emulsion, a microemulsion or a nanoemulsion, in particular oil-in-water or water-in-oil or multiple or silicone-based; a lotion, in particular in a glass or plastic bottle or in a measuring bottle or in an aerosol; an ampoule; a liquid soap; a dermatological cleansing bar; an ointment; a foam; an anhydrous product, preferably liquid, pasty or solid, for example in the form of a stick, in particular in the form of a lipstick.

The invention also relates to a composition administered orally to a human being or an animal, preferably a mammal, so as to obtain an effect chosen from the group consisting of an antioxidant, blood-cholesterol-lowering or blood-lipid-lowering effect, a stimulatory effect on the immune system, a hypoglycemic effect, in particular in the case of diabetes, and an effect consisting in preventing and/or treating and/or combating a pathology chosen from the group consisting of dyslipidemia, atherosclerosis, obesity, an obesity-related disease, a cardiovascular disease, metabolic syndrome, diabetes and hyperuricemia. Controlling the particle size of the chitin-glucan powder, in particular obtaining a powder with a fine particle size, advantageously allows better bioavailability of the product.

The invention also relates to a pharmaceutical or food supplement composition comprising, as active ingredient, at least one polysaccharide or one extract of fungal origin, as defined above.

The present invention also relates to a method of treating, preventing or combating a pathology, in particular that mentioned above, comprising the oral administration of an effective amount of a composition comprising at least one polysaccharide as defined in the description above and hereinafter, to an individual needing the latter.

The present invention also relates to a method for decreasing the weight or preventing or combating weight gain in a human being or an animal, and preferably a mammal. This method relates in particular to an esthetic care.

The present invention also relates to a method of cosmetic care, in particular for the body or the face, this care being advantageously chosen from the types of care mentioned above.

Thus, the present invention relates to the use of a product of the present invention, for the manufacture of a composition intended in particular to be used in one of the methods described above or for exerting one of the effects described above and hereinafter.

Those skilled in the art can readily determine, by conventional methods, the effective amounts of the products of the invention to be used. In the cosmetics or pharmaceutical industry, an effective amount of between 0.01% and 10% of the polysaccharide of fungal origin according to the present invention, by weight of the total composition, is advantageously used. Preferably, 0.05% to 5%, and more preferably from 0.1% to 2% by weight of the total composition is used. When applied topically, one or two applications a day is (are) advantageous.

Use is made of an effective amount of generally between 0.001% and 100% by weight of the product according to the invention, relative to the total weight of the composition to be administered in the form of a food supplement. If the products are administered in the form of gel capsules, granules or tablets, they can be used pure or at any other concentration, accompanied by other active components or excipients. If they are incorporated into foods, the concentration of product is less than 15%, and preferably less than 10%. It is advantageous to administer between 1 and 30 g of the product according to the invention, per day per individual, depending on the weight of the individual.

The invention also covers a) cohesive porous solid materials obtained by using fungal extracts in the form of particles with a fine and controlled particle size, and b) cohesive porous composite solid materials comprising a polymeric matrix which is synthetic or of natural origin (animal or plant), within which are distributed particles of fungal extracts in the form of particles with a fine and controlled particle size.

More generally, the cohesive porous materials of the present invention are obtained by using chitin polymers or chitin-glucan polymers in the form of particles with a fine and controlled particle size.

Also more particularly, the present invention covers cohesive porous composite materials of which the matrix is chitosan and within which are distributed particles of chitin and/or of chitin-glucan with a fine and controlled particle size.

For the purpose of the present invention, a “composite” material is an assembly of at least two materials.

For the purpose of the present invention, a “cohesive” material is a material characterized by its ability to remain stable and in the form of a monolith even under the action of external forces and stresses (compression, stretching, elongation, etc.), as opposed to friable material. Consequently, the cohesive material can be fashioned so as to give it a shape and a size suitable for its use (such as, for example, an implant of specific anatomical shape).

For the purpose of the present invention, a “porous” material is a material characterized by the presence of pores of which the size, the number, the morphology, the interconnectivity, the degree of isotropy/anisotropy, etc. are adjusted and controlled.

The prior art discloses numerous documents relating to the preparation of porous materials of natural polymers, such as chitosan or synthetic polymers such as polyurethane, PLA (polylactic acid), PGA (polyglycolic acid), PLGA (copolymer of lactic acid and of glycolic acid), etc. The prior art also discloses some documents relating to the preparation of compositions rich in fungal extracts containing chitin, or chitosan-glucan. However, none refer to the preparation of cohesive porous solid materials from these fungal extracts. For example, patent RU2086247 discloses a composition obtained starting from the mycelium of lower fungi (Aspergillus niger) and containing a chitosan-glucan complex with a view to preparing an anti-burn system. The method of preparation includes a step of washing and of alkaline treatment directly using the biomass, followed by a lyophilization step. However, the document does not refer to a product obtained in the form of a porous material. Sacchachitin is a composition rich in chitin extracted from the fruit of the fungus Ganoderma tsugae, the action of which on wound healing has been described by S H Su et al. (Development of fungal mycelia as skin substitutes: effects on wound healing and fibroblasts, Biomaterials 20, 61-68, 1999; Fungal mycelia as the source of chitin and polysaccharides and their applications as skin substitutes, Biomaterials 18, 1169-1174, 1997) and by Hung et al. (Cytotoxicity and immunogenicity of Sacchachitin and mechanism of action on skin wound healing, J Biomed Mater Res 56, 93-100, 2001).

JP2006273912 discloses a molded material composed of beta-glucan and of chitosan. It does not disclose that these materials are porous. Furthermore, they do not include chitin-glucan copolymers in their composition.

The prior art does not therefore disclose any document relating to cohesive porous materials obtained from fungal extracts, for instance chitin or a chitin-glucan copolymer in the form of particles with a fine and controlled particle size.

Porous materials comprising chitosan and a second compound have been widely described. Among many examples of a second compound, mention may be made of a synthetic polymer such as PGA (Biomaterials, 24 (2003), 1047-1057), or polyacrylic acid (Macromolecular Bioscience, 3(10), 2003, 540-545), natural polymers such as gelatin (Polymer International, 49(12), 2000, 1596-1599, CN1097980), collagen (WO0016817, KR2002017552, CN1406632, CN1387922, RU2254145), cellulose or silk (JP2000027027), oxidized cellulose (US2006172000), inorganic compounds such as hydroxyapatite (Journal of Biomaterial Science, Polymer edition, 13(9), 2002, 1021-1032).

US2003190346 covers a method for preparing a composite sponge made up of chitosan and chitin hydrogel, the particular form of the present invention not being envisioned. CN1485097 covers a method for preparing a sponge starting from chitosan/chitin. This document does not specify whether it is a composite material or materials made up of either chitosan or chitin. The abstract of this document discloses that the first step for preparing the sponge consists in solubilizing the starting material, which indicates that it involves preparing a sponge essentially of chitosan, since it is well known that chitin is insoluble, except under very specific conditions (dimethylacetamide-LiCl system).

No document discloses a composite material composed of chitosan and of chitin-glucan copolymer. In fact, it is well known to those skilled in the art that chitin-glucan copolymers are insoluble whatever the solvent. It is also well known that the methods for preparing a composite material, and in particular a biodegradable composite material, include, at the beginning, solubilization and homogeneous mixing of the solubilized compounds, before moving to the phase of removing the solvent with a view to preparing the solid material. The technical problem encountered in preparing a solid (in particular porous) chitin or chitin-glucan material or in preparing a solid (in particular porous) composite material composed of a polymer matrix, which is for example biodegradable, as first compound and of chitin or of chitin-glucan as second compound, is therefore how to solubilize, in a first step, the chitin or the chitin-glucan with a view to mixing it with the solution containing chitosan.

The present invention provides a technical solution to this problem by proposing the use of chitin and/or of chitin-glucan copolymer with a fine and controlled particle size in the form of a suspension, dispersion or emulsion that can be mixed with a solution of the biodegradable polymer serving as matrix.

Similarly, the present invention provides a technical solution to the preparation of a porous chitin or chitin-glucan solid by using a suspension, dispersion or emulsion containing the chitin or the chitin-glucan copolymer with a fine and controlled particle size.

The cohesive porous materials of the present invention cover
a) cohesive porous materials prepared from fungal extracts, preferably chitin polymers and/or chitin-glucan copolymers in the form of particles with a fine and controlled particle size, and more preferably chitin-glucan copolymers with a fine and controlled particle size;
b) cohesive porous composite materials comprising, as matrix, also known as dispersing agent, a polymer and as second compound, also known as dispersed agent, fungal extracts, preferably chitin polymers or chitin-glucan polymers in the form of particles with a fine and controlled particle size.

In particular, the invention also relates to:

A porous material comprising at least one fungal extract, preferably at least one chitin polymer and/or one chitin-glucan copolymer, in the form of particles with a particle size of less than 500 microns (μm).

Advantageously, the porous material comprises a fungal extract as defined above.

The invention also relates to a process for preparing a porous material, characterized in that it comprises a step of dispersing, or of emulsifying, or of suspending at least one fungal extract in the form of particles with a particle size of less than 500 microns, in a solvent, and then the elimination of this solvent and the obtaining of a porous material comprising the fungal extract.

The particles of chitin polymers or of chitin-glucan copolymers used for the porous material ‘a’ have a particle size of less than 250 μm, more preferably less than 90 μm, and even more preferably less than 63 μm. Preferably, use is made of particles of chitin-glucan copolymers having this fine and controlled particle size.

The invention also relates to a porous composite material comprising a matrix and a dispersed agent, said matrix, also known as dispersing agent, being at least one type of polymer, and the dispersed agent being at least one fungal extract, and preferably a chitin polymer or a chitin-glucan copolymer, in the form of particles with a particle size of less than 500 microns (μm).

Advantageously, the porous material comprises a fungal extract as defined above.

The porous composite material ‘b’ comprises particles of chitin or of chitin-glucan of the invention with a particle size of less than 250 μm, more preferably less than 90 μm, and even more preferably less than 63 μm. Preferably, use is made of particles of chitin-glucan copolymers having this fine and controlled particle size.

The invention covers a process for preparing a porous composite material comprising a matrix and an agent dispersed in the matrix, characterized in that it comprises (i) a step of solubilizing a polymer capable of forming the matrix of the porous composite material, (ii) a step of dispersing, or of emulsifying, or of suspending at least one fungal extract in the form of particles with a particle size of less than 500 microns, in the solution of polymer, (iii) a step of eliminating the solvent from the solution of polymer comprising the fungal extract, (iv) the obtaining of a composite material comprising the porous polymer forming the matrix and the fungal extract forming the dispersed agent.

The porous or porous composite material can form a layer or several layers of a composite material.

The polymeric matrix of the porous composite material ‘b’ may be of natural, animal or plant origin (an extracellular matrix (ECM) polymer). In particular, the polymers of natural origin (also known as biopolymers) may be chosen from the group consisting of glycosaminoglycans (GAGs), in particular hyaluronic acid or hyaluronate, chondroitin sulfate or heparin, collagenes, alginates, dextrans, chitosans, and mixtures thereof.

It would also be possible to choose synthetic polymers chosen from the group consisting of polyurethanes, polyacrylates, etc., or biodegradable synthetic polymers, in particular chosen from the group consisting of synthetic biodegradable polyesters such as homopolymers and copolymers based on lactic acid, glycolic acid, epsilon-caprolactone and p-dioxanone, or any other natural polyester, such as those of the polyhydroxyalkanoate family, for instance homopolymers and copolymers based on hydroxybutyrate, hydroxyvalerate, polyorthoesters, etc., or a polymeric matrix derived from a combination thereof or from the combination of natural polymers and of synthetic polymers. Preferably, the matrix is a biodegradable polymer.

Preferably, the biodegradable polymeric matrix is chitosan having any molecular mass and having any degree of acetylation.

The ratio by mass of the chitin or chitin-glucan polymer to the chitosan polymer (or biodegradable polymer) is between 5:95 and 95:5, preferably between 20:80 and 80:20. The preferred composition has a ratio by mass of between 40:60 and 60:40, preferably between 45:55 and 55:45, and more preferably of approximately 50:50.

The cohesive porous materials of the invention have any shape, any geometry and any size, and are preferably in the form of porous membranes, three-dimensional porous supports such as flat supports, porous foams, of porous microcarriers or beads, of porous fibers, of porous tubes, etc.

The cohesive porous materials have a density of at least 0.005 g/cm³.

The cohesive porous composite materials have a Young's modulus (which expresses the compression strength) of at least 0.05 MPa. The mechanical properties of the porous materials can be modulated, in particular depending on the size of the particles of the dispersed agent, on the ratio by mass of the matrix to the dispersed agent and the method of preparation, in particular on the concentration of the starting chitosan solution. Surprisingly, it has been noted that the ratio by mass of chitosan to the chitin-glucan copolymer of approximately 50:50 has the best compression strength.

The materials of the invention cover various pore sizes and total porosities, various morphologies (circular, elongated, fibrillar, etc.), various degrees of interconnectivity, or degrees of isotropy or anisotropy of the pores, various degrees of roughness of the pore walls, etc., as a function of the concentration of the suspension of chitin or chitin-glucan particles, of their size chosen from the fine and controlled particle size, of the ratio by weight of these particles to the polymer matrix and of the method of preparing these materials.

The cohesive porous materials of the invention have the advantages of providing chitin and chitin-glucan materials that can be easily handled and adapted, and are capable of being applied temporarily or permanently (in the form of dressings, pads, implants, patches, etc.), in cosmetic or pharmaceutical applications for which the beneficial effect of these compounds is desired, or in combination (for example impregnation, adsorption, absorption, inclusion in the pores, etc.) with other active agents.

Similarly, the porous composite materials of the invention have the advantage of combining the beneficial effects of the biodegradable polymer matrix and of the chitin and/or of the chitin-glucan copolymer with a fine and controlled particle size. Furthermore, the fine particles of chitin or of chitin-glucan can be anchored in the porous structure of the polymer matrix, conferring on it a rough structure, and characterized by a higher specific surface area. These characteristics can constitute an advantage for the use of this material in fields of application such as cell culture or tissue reconstruction, for which the stakes in terms of cell adhesion are high.

The method for preparing the cohesive porous materials ‘a’ and the cohesive porous composite materials ‘b’ comprises a first step of suspending, emulsifying or dispersing particles of chitin or of chitin-glucan with a fine and controlled particle size, either in water with a view to preparing the material ‘a’, or in a solution of a polymer matrix with a view to preparing the material ‘b’. In a second step, the mixture is subjected to drying or solvent elimination according to pyrogenic techniques known to those skilled in the art. Among the pyrogenic techniques, lyophilization, foaming in supercritical fluid (supercritical CO₂), pyrogenic salt extraction, immersion-precipitation, electrospinning and solid free-forming are suitable for carrying out this step. Preferably, the drying step is a lyophilization.

According to one particular embodiment of the invention for preparing the cohesive porous material ‘a’, the method comprises a first step (i) consisting in suspending the chitin polymer or preferably the chitin-glucan copolymer of the invention with a fine and controlled particle size, in water, according to a ratio by mass of between 05:95 and 30:70, preferably 05:95 and 20:80, followed by homogenization for at least 30 minutes with a view to preparing a paste. In a second step (ii), the paste is frozen by any freezing method, in particular by placing the paste in a freezer at −18° C. In a third step (iii), the frozen mixture is subjected to lyophilization so as to result in a cohesive porous material.

According to one particular embodiment of the invention for preparing a cohesive porous composite material ‘b’, the method comprises a first step (i) of solubilizing the biodegradable polymer matrix in a solvent and according to experimental conditions allowing complete solubilization thereof. Preferably, the chitosan is solubilized in a proportion of 1% to 10% in a solution of dilute acid at a concentration of between 0.5% and 5%, preferably between 0.5% and 2%. Among the acids that can be used for this step, inorganic acids such as, for example, hydrochloric acid, hydrofluoric acid, phosphonic acid, etc., or organic acids such as, for example, acetic acid, formic acid, lactic acid, glycolic acid, gluconic acid, citric acid, succinic acid, glutamic acid, etc., are suitable. In a second step (ii), the chitin or chitin-glucan particles with a fine and controlled particle size are dispersed in the solution containing the polymer matrix, homogenized for at least one minute and then poured into a mold chosen according to the size, the geometry and the properties of the porous composite material to be prepared. The ratio by mass of the matrix polymer to the dispersed agent is between 10:90 and 90:10. In a third step (iii), the mixture is subjected to freezing by any freezing technique. In a final step (iv), the frozen mixture is subjected to lyophilization. The composite material obtained is porous. The density, the size and the morphology of the pores, and the mechanical properties, in particular the compression strength properties, of the material can be adjusted, according to this embodiment of the invention, as a function of the concentration of the polymer matrix, of the ratios by mass of the polymer matrix to the dispersed agent, the nature of the matrix solvent, the type of mold, the filling volume of this mold, and the freezing conditions.

In the figures:

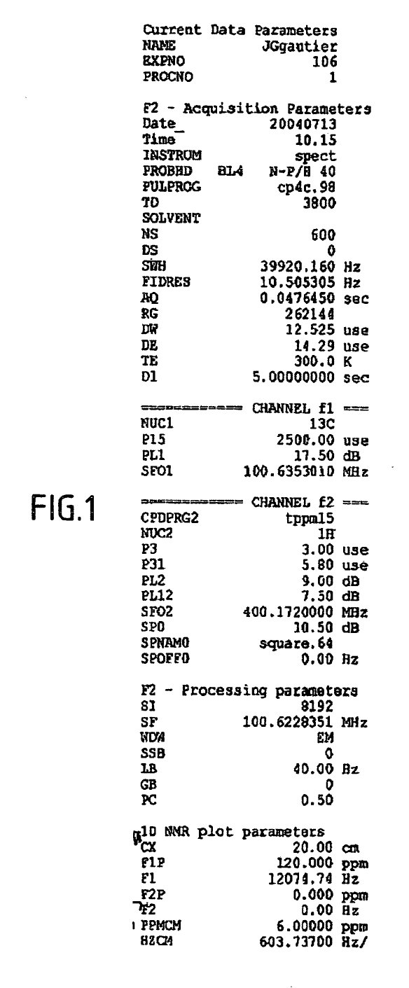

FIG. 1 represents the conditions for recording the solid-phase carbon 13 nuclear magnetic resonance (¹³C-NMR) spectrum of a chitin-glucan copolymer.

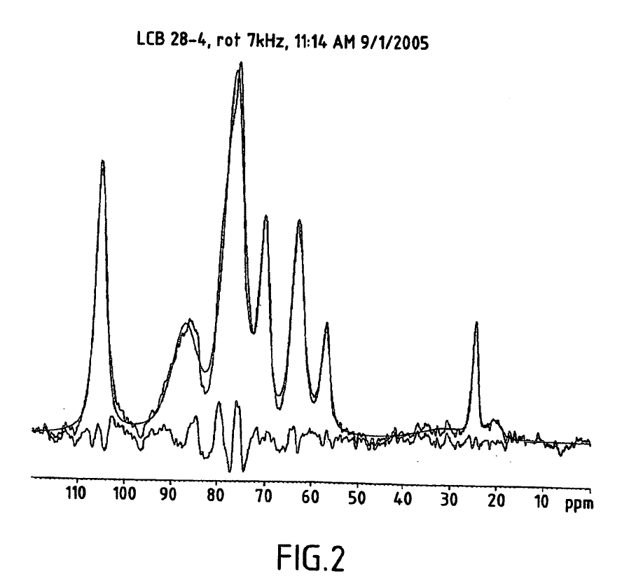

FIG. 2 represents the solid-phase ¹³C-NMR spectrum of a chitin-glucan copolymer.

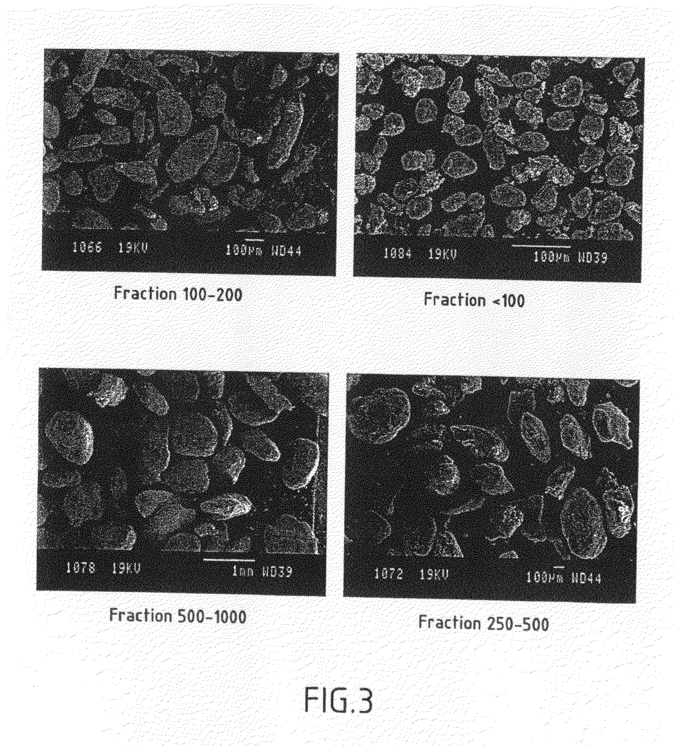

FIG. 3 represents four scanning electron microscopy photographs of the chitin-glucan particles (batch L26) according to the present invention, and in particular of the fractions of size 100-200 μm (fraction 100-200), of size <100 μm (fraction<100), of size 500-1000 μm (fraction 500-1000), and of size 250-500 μm (fraction 250-500).

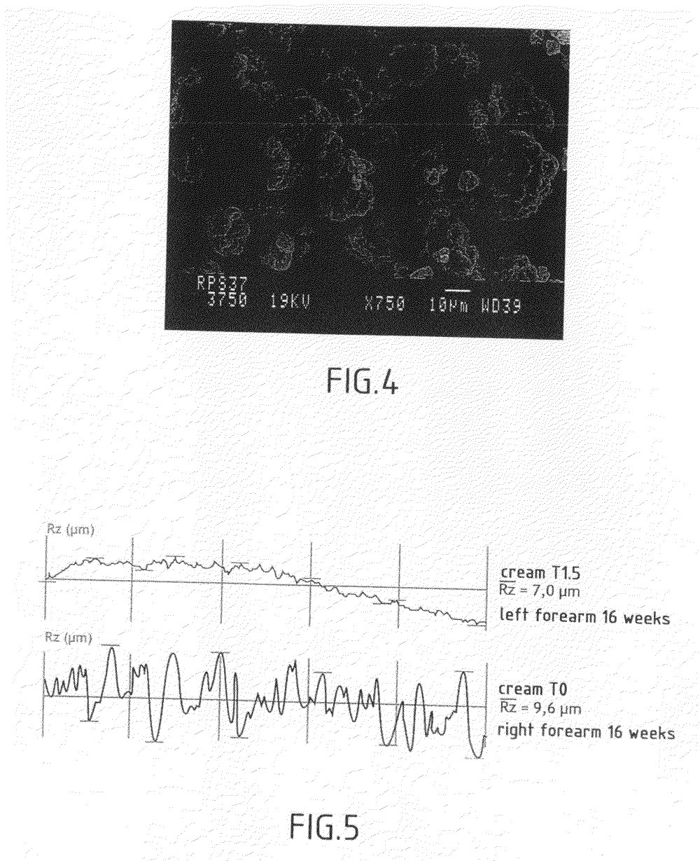

FIG. 4 represents a scanning electron microscopy photograph of the particles according to the present invention (batch L32), after drying by means of a spraying process (magnification×750).

FIG. 5 represents an optical profilometry graph, bearing the height of the furrows (Rz) as a function of the distance on the skin, obtained after 16 weeks on one individual (left forearm: chitin-glucan-based cream T1.5; right forearm: placebo cream T0). The graphs show that the microcontours are significantly reduced (mean value Rz 7.0 μm with chitin-glucan versus 9.6 μm with the placebo) and that the skin is more taut.

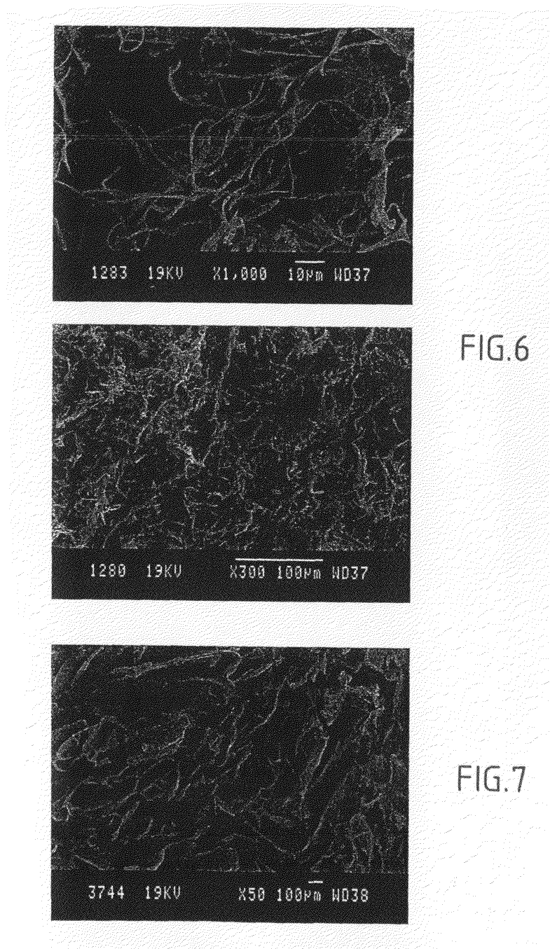

FIG. 6 represents two scanning electron microscopy photographs of a porous chitin-glucan material.

FIG. 7 represents a scanning electron microscopy photograph of a mixed porous material of chitin-glucan and of chitosan (chitin-glucan/chitosan 10:90, m/m) in longitudinal section.

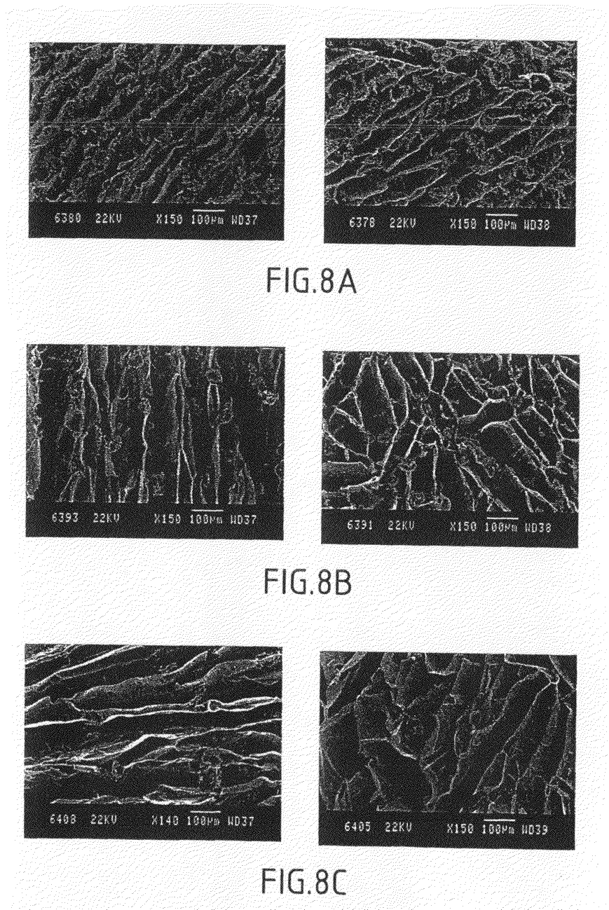

FIGS. 8A-C represent scanning electron microscopy photographs of a composite porous material of chitin-glucan and of chitosan, obtained for three samples (FIG. 8A: chitosan/chitin-glucan proportion (m/m) of 25/75, FIG. 8B: chitosan/chitin-glucan proportion (m/m) of 50/50 and FIG. 8C: chitosan/chitin-glucan proportion (m/m) of 75/25). For each of the samples, the size of the chitin-glucan particles is less than 63 μm. The photographs on the left represent a longitudinal section and those on the right a transverse section.

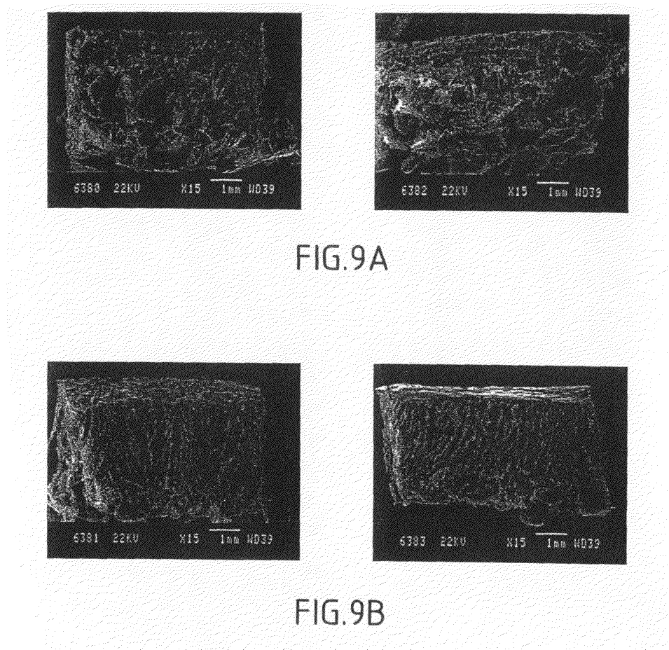

FIGS. 9A and 9B represent scanning electron microscopy photographs of a composite porous material of chitin-glucan and of chitosan, obtained for four samples obtained with chitin-glucan particles having a particle size of greater than 250 μm.

FIG. 9A left-hand photo: chitosan/chitin-glucan proportion (m/m) of 25/75 and grain size of between 250 and 500 μm.

FIG. 9A right-hand photo: chitosan/chitin-glucan proportion (m/m) of 25/75 and grain size of between 500 and 1000 μm.

FIG. 9B left-hand photo: chitosan/chitin-glucan proportion (m/m) of 50/50 and grain size of between 250 and 500 μm.

FIG. 9B right-hand photo: chitosan/chitin-glucan proportion (m/m) of 50/50 and grain size of between 500 and 1000 μm.

Other objectives, characteristics and advantages of the invention will emerge clearly to those skilled in the art upon reading the explanatory description which refers to examples that are given only by way of illustration and can in no way limit the scope of the invention.

The examples are an integral part of the present invention and any characteristic that appears to be new in relation to any prior art based on the description taken in its entirety, including the examples, is an integral part of the invention in terms of its function and its generality.

Thus, each example has a general scope.

Furthermore, in the examples, all the percentages are given by weight, unless otherwise indicated, and the temperature is expressed in degrees Celsius unless otherwise indicated, and the pressure is atmospheric pressure unless otherwise indicated.

### EXAMPLES

The process for obtaining the chitin-glucan copolymers obtained below is described in patent applications WO 03/068824 and FR 05.07066.

### Series of examples ‘A’ relating to the preparation of a chitin-Glucan Copolymer and of Particles Thereof with a Variable and Controlled Particle Size

### Example A1

### Preparation of a Chitin-Glucan Copolymer from the Mycelium of Aspergillus niger

A mass of 50 kg (dry weight) of wet Aspergillus niger mycelium is suspended in a 0.5 N solution of hydrochloric acid and then filtered. The solid matter is then suspended in a 1 N solution of sodium hydroxide and then filtered. The solid matter is washed 4 times with water, and then filtered using a filter press and dried using a conical drier. It is subsequently suspended in ethanol and then filtered and dried. Approximately 15 kg of chitin-glucan are obtained (batch L25).

The molecular characteristics and the composition of eight batches of chitin-glucan obtained according to this process are given in Table 1.

The chitin/glucan ratio by mass is calculated from the solid-phase carbon 13 nuclear magnetic resonance (NMR) spectrum recorded under the conditions indicated in FIG. 1 according to the method described briefly below. The spectrum of the chitin-glucan compound (batch L28) is shown in FIG. 2. The proportion of beta-glucan is determined from the area of the following four resonance bands: 104 ppm (carbon 1 of the chitin and of the beta-glucan), 23 ppm (CH₃ carbon of the chitin), 55 ppm (carbon 2 of the chitin) and 61 ppm (carbon 6 of the chitin and of the beta-glucan), taking pure chitin as reference. For example, the calculation can be done according to formula 1, where I′ is the area of the signals of the carbons, and where [ ]_{CG} indicates the value of the ratio for the chitin-glucan analyzed and []_{C} the value for the reference chitin. C1 is the carbon 1 of the chitin and of the beta-glucan and C2 is the carbon 2 of the chitin.

$$Glucan ( mol \% ) = \frac{[ \frac{{I}^{′} ( C 1 )}{{I}^{′} ( C 2 )} ] CG - [ \frac{{I}^{′} ( C 1 )}{{I}^{′} ( C 2 )} ] C}{[ \frac{{I}^{′} ( C 1 )}{{I}^{′} ( C 2 )} ] CG} \times 100 \qquad ( formula 1 )$$

The chitin/glucan ratio by mass of the 8 batches of chitin-glucan of Table 1 is on average 39:61±2 (m/m).

The proportion of D-glucosamine (NGIc) units, expressed as molar % of the chitin part, can be estimated from the NMR spectrum, as described by Heux et al. [Heux L, Brugnerotto], Desbrieres J, Versali M F & Rinaudo M. (2000) Solid state NMR for determination of the degree of acetylation of chitin and chitosan. Biomacromolecules 1:746]. The proportion of D-glucosamine units is determined by potentiometric titration with sodium hydroxide, in suspension in an excess of hydrochloric acid.

The microbiological quality of the chitin-glucan (batch L26) and the results of searching for pathogenic agents are given in Table 2.

The size distribution of the chitin-glucan powder (batch L25) is given in Table 3.

TABLE A1.1
Molecular characteristics and composition of various batches of
chitin-glucan copolymer

| | Chitin-glucan | NGlc | | | | Heavy |
|---|---|---|---|---|---|---|
| | ratio | (titration) | Ash | Proteins | Lipids | metals |
| Batch | (m/m) | mol % | (%) | (%) | (%) | (ppm) |
| L25 | 41:59 ± 4* | 0 | 1.5 | 4.2 | 0.5 | <LQ** |
| L26 | 36:64 ± 5 | 0 | 0.4 | 4.6 | 0 | <LQ |
| L27 | 42:58 ± 7 | 0 | 1.3 | 3.5 | 0 | <LQ |
| L28 | 39:61 ± 7 | 0 | 1.5 | 2.5 | 2.1 | <LQ |
| L29 | 40:60 ± 6 | 0 | 1.7 | 3.1 | 0.1 | <LQ |
| L30 | 37:63 ± 1 | 0 | 1.9 | 1.5 | 1.2 | 8.7 |
| L31 | 40:60 ± 4 | 0 | 2.5 | 4.3 | 1.3 | <LQ |

*standard deviation over the result of 4 calculations of the chitin-glucan ratio;
**LQ: limit of sensitivity of the Ion Coupled Plasma method of analysis (5.3 ppm)

TABLE A1.2
Microbiological quality of a batch of chitin-glucan (L26)

| | Number of |
|---|---|
| | microorganisms/g |
| Total mesophilic aerobic | <20 cfu/g |
| microorganisms | |
| Aerobic spores | <10 cfu/g |
| Yeasts and moulds | <20 cfu/g |
| Pathogenic agents | |
| Enterobacteriaceae | Absence |
| Escherichia coli | Absence |
| Staphylococcus coagulase+ | Absence |
| Pseudomonas spp | Absence |
| Salmonella spp | Absence |

It is thus understood from the above tables that the copolymer according to the present invention has a high degree of purity.

TABLE A1.3
Size distribution of a batch of chitin-glucan (L25)

| Cumulative | | Non-cumulative | Size |
|---|---|---|---|
| proportion | Diameter | proportion (%, | range |
| (%, m/m) | (μm) | m/m) | (μm) |
| 100% | Less than 1000 μm | 13% | 710-1000 |
| 87% | Less than 710 μm | 20% | 500-710 |
| 67% | Less than 500 μm | 16% | 355-500 |
| 51% | Less than 355 μm | 14% | 250-355 |
| 37% | Less than 250 μm | 11% | 180-250 |
| 26% | Less than 180 μm | 13% | 125-180 |
| 14% | Less than 125 μm | 11% | 90-125 |
| 3% | Less than 90 μm | 3% | 0-90 |

It is understood from this table that the size distribution of the particles obtained according to the process of example 1 is very broad.

### Example A2

### Preparation of Chitin-Glucan Powder with a Variable Particle Size, by Milling

In order to reduce the particle size in a controlled and variable manner, 15 kg of chitin-glucan, obtained according to example 1 (batch L25), are milled in a hammer mill (Fitzmill model D, Fitzpatrick) equipped with filters of various geometries and with a screen size of 20 to 100 mesh (references A, B, C, D in table 1). Four batches of chitin-glucan powder are thus obtained, the size distribution of which, determined by screening on calibrated screens and gravimetric analysis, is that indicated in table 1.

TABLE A2.1
Size distribution of a batch of chitin-glucan obtained by milling
using various types of screens

| Batch of | Milling | Distribution | % |
|---|---|---|---|
| chitin-glucan | screen | (μm) | (m/m) |
| L25D | 100 mesh | 500-1000 | 19% |
| | | 250-500 | 25% |
| | | 200-250 | 1% |
| | | 100-200 | 28% |
| | | <100 | 27% |
| L25A | 65 mesh | 500-1000 | 25% |
| | | 250-500 | 41% |
| | | 100-250 | 25% |
| | | <100 | 7% |
| L25C | 40 mesh | 250-500 | 44% |
| | | 200-250 | 14% |
| | | 100-200 | 29% |
| | | <100 | 11% |
| L25B | 20 mesh | 500-1000 | 25% |
| | | 250-500 | 40% |
| | | 200-250 | 10% |
| | | 100-200 | 17% |
| | | <100 | 7% |

It is understood from this table that particles of small size, below 200 μm for example, are readily obtained by milling the chitin-glucan powder with a fine screen, for example of 100 or 65 mesh.

The batches of chitin-glucan milled with the various screens are combined by fraction, by screening on calibrated screens, and the powders are observed by scanning electron microscopy after platinum metallization (FIG. 4). The length and the width of the particles is calculated after analysis of about one hundred particles per sample (table A2.2).

TABLE A2.2
Dimensions of the fractions of milled chitin-glucan (batch L25)
observed by SEM

| Size range | Length | Width |
|---|---|---|
| (μm) | (μm) | (μm) |
| <50 | 54 ± 11 | 37 ± 9 |
| 90-100 | 134 ± 26 | 80 ± 20 |
| 100-200 | 207 ± 66 | 113 ± 33 |
| 200-250 | 358 ± 85 | 190 ± 58 |
| 250-500 | 514 ± 118 | 329 ± 88 |
| 500-1000 | 1029 ± 245 | 683 ± 132 |

It is understood from this table that the hammer-milling technique produces ovoid, non-spherical particles, as revealed by the scanning electron microscopy observation in FIG. 4.

The 100-200 μm fraction of batch L25 is the product used to prepare the test cream (reference T1.5) of example C.1.

### Example A3

### Preparation of Chitin-Glucan Powders with a Fine and Controlled Particle Size, Obtained by Spraying Solvated Chitin-Glucan

In order to obtain a chitin-glucan powder with a fine particle size, with particles of spherical geometry, a paste containing 0.15 kg of chitin-glucan (batch L25) in 3.75 liters of water is prepared using a mixer. The paste is spray-dried at a temperature of 200° C. 0.15 kg of a powder for which the cumulative size distribution is that given in table A3.1 is obtained. The photograph in FIG. 5 represents the particles observed by scanning electron microscopy.

TABLE A3.1
Size distribution of a batch of chitin-glucan (L25) after spray drying

| % Proportion | |
|---|---|
| (cumulative) | Diameter (μm) |
| 96% | Less than 250 μm |
| 93% | Less than 180 μm |
| 73% | Less than 125 μm |
| 35% | Less than 90 μm |

It is understood from this result that the solvated chitin-glucan spraying technique makes it possible to obtain predominantly a fine and homogeneous particle size, 73% of the particles having a diameter of less than 125 μm.

### Example A4

### Preparation of Chitin-Glucan Powders with a Fine and Controlled Particle Size Obtained by Drying Solvated Chitin-Glucan in a Nütsche Filter

In order to obtain a fine chitin-glucan powder, a paste containing 50 kg of chitin-glucan solvated in ethanol is dried in a Nütsche filter at a temperature of 60° C. for 12 hours. 50 kg of a chitin-glucan powder (batch L16) for which the cumulative size distribution is that given in table A4.1 are obtained. The tapped (or packed) density of the chitin-glucan powder thus obtained, determined according to the method of the European Pharmacopeia 2.9.15, is 0.71 g/cm³.

TABLE A4.1
Size distribution of a batch of chitin-glucan after drying (batch
L16)

| % Proportion | |
|---|---|
| (cumulative) | Diameter (μm) |
| 75% | Less than 355 μm |
| 70% | Less than 250 μm |
| 67% | Less than 180 μm |
| 60% | Less than 125 μm |
| 30% | Less than 90 μm |

It is understood from this result that the technique of drying the chitin-glucan by means of a Nütsche filter using chitin-glucan solvated in ethanol makes it possible to obtain predominantly a fine and homogeneous particle size, with 60% of the particles having a diameter of less than 125 μm.

The powder obtained is fractionated by screening so as to select therefrom the particles having a size of less than 90 μm. This fraction is used to prepare the formulations of examples 14 to 20. This fraction less than 90 μm, observed by scanning electron microscopy, reveals an average size, determined by image analysis, of 43±18 μm. The tapped density of the fraction of size less than 90 μm, determined according to the method of the European Pharmacopeia 2.9.15, is 0.61 g/cm³.

### Example A5

### Preparation of a Chitin-Glucan Powder with a Particle Size of Less than 125 μm, by Milling and Screening

In order to obtain a powder with a particle size of less than 125 μm, a batch of dried chitin-glucan [L07073CG] is milled in a disk mill, and then screened on a calibrated screen with a mesh size equal to 125 μm (or 120 mesh) introduced into an industrial screening device. The particle-size dispersion obtained after each milling cycle is given in table A5.1.

TABLE A5.1
Particle-size dispersion of the chitin-glucan powder before
milling

| | | | Cumulative |
|---|---|---|---|
| | Cumulative | Cumulative | proportion |
| | proportion | proportion | (%, m/m) after |
| Diameter | (%, m/m) | (%, m/m) after | second milling |
| (μm) | before milling | first milling cycle | cycle |
| Less than | 100% | 100% | 100% |
| 1000 μm | | | |
| Less than | 27% | 97% | 100% |
| 250 μm | | | |
| Less than | 14% | 87% | 94% |
| 180 μm | | | |
| Less than | 3% | 52% | 73% |
| 125 μm | | | |
| Less than | 0% | 34% | 53% |
| 90 μm | | | |
| Less than | 0% | 14% | 27% |
| 50 μm | | | |

The importance and the efficiency of the milling and screening step for increasing the particle-obtaining yield is understood by virtue of these tables. A much higher proportion of powder with a particle size of less than 125 μm is obtained after the first milling. This proportion is further increased after a second milling. This example shows a high proportion (more than 50% m/m) of particles having a particle size of less than 90 μm, and even a large proportion of particles having a particle size of less than 50 μm (more than 25% m/m).

### Series of Examples B

### Examples of preparation of Chitin-Glucan-Based Simple Creams Intended for Tolerance Studies and Results of the Chitin-Glucan-Tolerance Studies

The objective of these examples is in particular to show the innocuousness of a chitin-glucan copolymer in the form of particles (whatever the size thereof). These simple compositions are limited to being a support for spreading of the chitin-glucan copolymer on the skin with a view to the study of tolerance.

### Example B1

### Preparation of a Chitin-Glucan-Based Simple Cream Intended for Tolerance Studies

An emulsion-type cream was prepared based on chitin-glucan (batch L31), the latter being incorporated into the aqueous phase, the paste obtained being homogenized for approximately 1 h using a knife mill (Ultraturax, 10 000 rpm): a simple emulsion containing the chitin-glucan at a varying concentration of from 0 to 2.5% (table B1.1). A placebo formulation (reference C0) was prepared under the same conditions without chitin-glucan.

TABLE B1.1
Composition of the simple cosmetic creams based on chitin-glucan at a
concentration of X % (references CX)

| | | Concentration |
|---|---|---|
| Ingredients | INCI | (%) |
| Water (QS ad | Aqua | qs |
| 100%) | | |
| Simulgel EPG | Sodium | 3% |
| | acrylate/acryloyldimethyl | |
| | taurate copolymer, | |
| | Polyisobutene, | |
| | Caprylyl capryl glucoside | |
| Nexbase 2006 | Hydrogenated | 10% |
| FG | polydecene | |
| Chitin-glucan | Chitin, Beta-glucan | X % |
| (powder, L31) | | X = 0 or 0.5 or |
| | | 1.0 or 1.5 or |
| | | 2.0 or 2.5% |
| Phenochem | Phenoxyethanol (60%), | 0.8% |
| | Methylparaben, Ethylparaben, | |
| | Propylparaben, Butylparaben, | |
| | Isobutylparaben | |
| Disodium EDTA | Disodium EDTA | 0.05% |

C0, C0.5, C1.0, C1.5, C2.0 and C2.5 are thus prepared.

### Example B2

### Study of the Cutaneous Tolerance and of the Hydrating Capacity of a Simple Cream Based on Chitin-Glucan at Varying Concentrations

Protocol—A series of 4 creams of oil-in-water emulsion type, containing a chitin-glucan concentration of 0.5% to 2%, the composition of which is given in example B1, was tested by application to the forearm of 13 volunteers, with an average age of 46 (from 32 to 61), whose skin is sensitive at this site. The creams are compared with a chitin-glucan-free placebo cream (reference C0). The products were applied twice a day on delimited zones on each forearm (3 zones of 6.25 cm²).

Three types of biometrological measurements were carried out every 2 weeks for 6 weeks in order to investigate the tolerance and the effectiveness of the products. The initial clinical examination revealed nothing abnormal in any of the individuals. The evaluations were carried out in the morning, with a gap of at least 10 hours after the last application of the products.

Biometrological measurements—Any possible erythema, a sign of irritation, was sought by corneometry, by measuring the parameter a* given by reflectance colorimetry evaluated according to the CIE standards L*a*b* (Minolta Chroma Meter® CR200). Any impairment of the barrier function was sought by measuring the transepidermal water loss (TEWL) in g/m²/hr (Tewameter®, C+K Electronic). The TEWL is 5 to 7 g/m²/hr for normal skin, and 15 to 20 g/m²/hr for dry skin. The water impregnation dynamics of the horny layer under occlusion was measured. The “rate of water accumulation” (RWA) was evaluated over a period of 30 seconds (RWA 30) by the ratio of the difference between the values at T30s and at T0s to the value at T0s. The higher the value, the drier the horny layer and the more permeable it is to water, allowing it to leak without taking it up, which is the equivalent of the TEWL.

Statistics—At each evaluated time, the inter-product comparison was carried out by means of Friedman's non-parametric test followed by Dunn's test. For each product, the comparison over time was carried out according to the same methods. For each parameter, significant differences between the values obtained at the various times were sought by means of Friedman's non-parametric pair test followed by Dunn's test.

Results—The values of a*, TEWL and RWA30, measured at least 10 hours after application of the 5 creams at times 0, 2, 4 and 6 weeks, are listed in table B2.1. Up to the maximum concentration of chitin-glucan studied, which was 2.0%, no sign of erythema, of irritation or of impairment of the cutaneous barrier was observed throughout the 6-week period, as demonstrated by the a* and TEWL values which do not increase. It was, on the other hand, demonstrated that the rate of water accumulation at the horny surface under occlusion (RWA30) is significantly lower after application of the chitin-glucan-based creams, which indicates a better capacity of the skin to retain water at the surface. This effect is observed from the second week of application onward, for the concentrations of 1.5% and 2.0%, and from the fourth week onward for the concentrations of 0.5% and 1.0%.

TABLE B2.1
Results of reflectance colorimetry (a*), transepidermal water loss
(TEWL) and water impregnation dynamics (RWA30, rate of water
accumulation over 30 seconds)

| | Reference | | | |
|---|---|---|---|---|
| Time | of the | | TEWL | |
| (week) | cream | a* | g/m²/hr | RWA30 |
| Week 0 | C0 | 5.36 ± 0.60 | 5.52 ± 0.72 | 1.05 ± 0.48 |
| | (placebo) | | | |
| | C0.5 | 5.39 ± 0.62 | 5.50 ± 0.60 | 1.11 ± 0.49 |
| | C1.0 | 5.39 ± 0.60 | 5.49 ± 0.70 | 1.15 ± 0.46 |
| | C1.5 | 5.42 ± 0.55 | 5.41 ± 0.66 | 1.14 ± 0.50 |
| | C2.0 | 5.38 ± 0.56 | 5.49 ± 0.62 | 1.16 ± 0.49 |
| Week 2 | C0 | 5.4 ± 0.5¹ | 5.45 ± 0.66 | 0.96 ± 0.48¹ |
| | (placebo) | | | |
| | C0.5 | 5.4 ± 0.5¹ | 5.42 ± 0.69 | 1.01 ± 0.53¹ |
| | C1.0 | 5.4 ± 0.4¹ | 5.42 ± 0.63 | 0.88 ± 0.46 |
| | C1.5 | 5.3 ± 0.5¹ | 5.36 ± 0.56 | 0.74 ± 0.47² |
| | C2.0 | 5.1 ± 0.4² | 5.25 ± 0.60 | 0.65 ± 0.42² |
| Week 4 | C0 | 5.47 ± 0.49¹ | 5.65 ± 1.05 | 0.91 ± 0.42¹ |
| | (placebo) | | | |
| | C0.5 | 5.49 ± 0.41¹ | 5.58 ± 0.86 | 0.83 ± 0.46 |
| | C1.0 | 5.42 ± 0.38¹ | 5.57 ± 0.88 | 0.70 ± 0.39² |
| | C1.5 | 5.24 ± 0.31² | 5.53 ± 0.85 | 0.62 ± 0.36² |
| | C2.0 | 5.20 ± 0.31² | 5.42 ± 0.96 | 0.57 ± 0.38² |
| Week 6 | C0 | 5.42 ± 0.67¹ | 5.48 ± 0.76¹ | 0.83 ± 0.36¹ |
| | (placebo) | | | |
| | C0.5 | 5.39 ± 0.55¹ | 5.47 ± 0.68¹ | 0.76 ± 0.42¹ |
| | C1.0 | 5.38 ± 0.54¹ | 5.38 ± 0.71 | 0.68 ± 0.35¹ |
| | C1.5 | 5.32 ± 0.53¹ | 4.98 ± 1.45² | 0.56 ± 0.35² |
| | C2.0 | 5.18 ± 0.49² | 5.21 ± 0.49² | 0.46 ± 0.34² |

^{1,2}the values bearing a different superscript number are significantly different (p < 0.02) from one another, within a series of measurements at the same time.

### Example B3

### Study of the Tolerance, by Corneoxenometry on the Horny Layer in Vitro, of Simple Creams Based on Chitin-Glucan in Varying Concentration

Protocol—Cyanoacrylate surface biopsies were taken from the forearms of 15 healthy volunteers in order to sample the horny layer. The creams containing chitin-glucan in varying concentration of from 0 to 2% are those of example 5 (from 0.5% to 2.5%). They were diluted with water to 50:50 (v/v) in order to be able to guarantee intimate contact between the products and the horny layer. The solutions were deposited onto the horny layer samples for 2 hours. At the end of this contact, the samples were thoroughly rinsed with water. After drying, they were stained for 1 min with an alcoholic solution of basic fuschin and toluidine blue. After rinsing with water and drying, the color of each sample was measured by reflectance colorimetry for the L* and Chroma C* mode. The difference in L* and C* corresponds to the calorimetric index of mildness (CIM). A CIM of greater than 40 indicates a product that is very mild for the skin.

Results—The CIM values calculated for the 5 creams are listed in table B3.1. They are greater than 70 irrespective of the concentration of chitin-glucan in the formulation applied, and they reflect the excellent tolerance of the 6 products.

TABLE B3.1
Colorimetric index of mildness (CIM = L* − C*) of the horny
layer after 2 hours of contact with the solutions containing the
chitin-glucan-based creams, diluted 2-fold

| N = 15 | | | | | |
|---|---|---|---|---|---|
| Reference | C0 | C0.5 | C1.0 | C1.5 | C2.0 |
| Mean CIM | 78.6 | 79.3 | 74.0 | 78.4 | 77.6 |
| Standard | 6.7 | 4.3 | 8.3 | 5.4 | 7.1 |
| deviation | | | | | |
| Median CIM | 75.0 | 80.8 | 75.0 | 80.0 | 78.1 |

### Example B4

### Clinical Evaluation of the Irritant and Sensitizing Potential of the Chitin-Glucan

The clinical evaluation of the sensitizing potential and of the hypoallergenicity of the chitin-glucan is carried out according to the Maibach-Marzulli protocol, on 50 volunteers with normal skin (37±2 years old), for 6 weeks. A paste with a concentration of 10% is prepared by dispersing the chitin-glucan (L25) in water. The paste is applied to the skin by means of a Finn Chamber® occlusion patch. Possible signs of erythema, cedemia, dryness and the appearance of vesicules are observed in order to characterize the irritant potential (induction phase) and the sensitizing potential (challenge phase) of the product.

In view of all the observations, the chitin-glucan is considered to be non-irritant and non-sensitizing. It can therefore bear the claim “hypoallergenic”.

### Example B5

### Primary Skin Irritation by the Chitin-Glucan, in vivo Study on Volunteers

The primary irritation by the chitin-glucan on the skin is evaluated by applying the chitin-glucan (L25) in the form of a 10% aqueous paste (as in example B4), applied to the skin in a Finn Chamber® occlusion patch, for 24 hours. The study is carried out on 10 individuals. The observations aimed at detecting signs of erythema, oedemia and structural modifications of the skin are made 30 minutes and 24 hours after the detachment of the patch, under dermatological control. All the results of the study indicate that the chitin-glucan (dispersed at 10% in water) can be classified as non-irritant to the skin.

### Example B6

### Ocular Irritation by the Chitin-Glucan (in vitro, HE7-CAM Method)

The ocular irritation by the chitin-glucan (L25) is evaluated by means of the HET-CAM test on embryonated hen's egg chorioallantoic membrane (hen's egg test-chorioallantoic membrane), according to Luepke et al. [Fd Chem Toxic 23, 287, 1985], officially recognized as an alternative to animal experimentation (OJ of 26/12/1996). The chitin-glucan dispersed at 5% in water is deposited at the surface of the membrane and brought into contact for 20 seconds. The test is repeated on four eggs. The 5% chitin-glucan obtains the lowest score, classifying it as virtually non-irritant to the hen's egg chorioallantoic membrane.

### Series of Examples C

### Preparation of Suspensions, Dispersions, and Emulsions Based on Chitin-Glucan with a Fine and Controlled Particle Size

### Example C1

### Chitin-Glucan-Based Day Cream Intended for Studying the Properties On Elderly Volunteers

TABLE C1.1
Comparison of the placebo test cream (reference T0) and test cream
based on chitin-glucan (batch L25) at a concentration of 1.5%
(reference T1.5)

| | | | | Concen- |
|---|---|---|---|---|
| | | | | tration |
| Phase | Ingredient | INCI | Function | (%) |
| A | Water | Aqua | | qs |
| | Fucogel | Biosaccharidegum-1 | Moisturizer | 5 |
| | 1000PP | | | |
| | Glycerol | Glycerin | Cold- | 2 |
| | | | stabilizer | |
| | Rice NS | | Feel | 1 |
| | Neocare | Cetearyl glucoside | Emulsifier | 0.2 |
| | CG90 | | | |
| | Keltrol T | Xanthan gum | Prevention of | 0.1 |
| | | | creaming | |
| | Veegum HS | Aqua, Magnesium | Prevention of | 20 |
| | 50% solution | aluminum silicate | creaming | |
| | Euxyl K300 | Phenoxyethanol, | Preservative | 0.8 |
| | | Methylparaben, | | |
| | | Ethylparaben, | | |
| | | Propylparaben, | | |
| | | Butylparaben | | |
| B | Inutec Sp1 | Inulin lauryl | Stabilizer | 0.3 |
| | | carbamate | | |
| | Sabonal | Cetearyl alcohol | Thickener | 3.5 |
| | C1618 50/50 | | | |
| | Shea butter | Butyrospermum | Emollient | 1.5 |
| | | Parkii | | |
| | Stearic acid | Stearic acid | Matting agent | 2 |
| | Antiox_cos | Soya glycine, | Antioxidant | 0.5 |
| | | Tocopherol, | | |
| | | ascorbyl palmitate, | | |
| | | Lecithin | | |
| | Jojoba oil | Buxus chinensis | Emollient | 4 |
| | Borage oil | Borago officinalis | Emollient | 5 |
| | Macadamia | Macadamia | Emollient | 5 |
| | nut oil | ternifolia | | |
| | Neoderm | Cetearyl | Improves | 1 |
| | CSO | ethylhexanoate | applicability | |
| | Neoderm | Cetearyl | Improves | 1 |
| | CSN | isononanoate | applicability | |
| | Neoderm | Pentaerythrithyl | Texturing | 1.5 |
| | PTC | Tetracaprylate/ | effect | |
| | | Caprate | | |
| | Velvesil DM | Dimethicone, | Feel | 4 |
| | | Cetearyl | | |
| | | dimethicone | | |
| | | crosspolymer | | |
| | SF1256 | Cyclopentasiloxane, | Prevents | 1 |
| | | Cyclohexasiloxane | whitening of | |
| | | | the skin on | |
| | | | application | |
| C | Chitin- | Chitin, Beta-glucan | Active agent | X |
| | glucan (L25 | | | X = 0 |
| | fraction | | | (reference |
| | 100-200 μm) | | | T0) or 1.5 |
| | | | | (reference |
| | | | | T1.5) |
| D | Cool Woman | Fragrance | Fragrance | 0.5 |
| | 341992-N | | | |

Procedure for Preparing the Test Cream (reference T1.5)

The ingredients of phase A are mixed at 80° C., and then the ingredients of phase B are mixed at 75° C. Phase B is added to phase A, and the mixture is homogenized with a mixer, and then left to cool. The ingredients C and S are finally added at 40° C.

TABLE C1.2
Characteristics of the T0 and T1.5 creams

Cream reference

| | T0 | T1.5 |
|---|---|---|
| pH | ND | 5-7 |
| Viscosity (mPa · s) | 18000 | 19000 |
| Density | ND | 1000 |

### Example C2

### Influence of the Particle Size of the Chitin-Glucan on the Preparation and the Sensory Characteristics of Chitin-Glucan-Based Cosmetic Creams Containing Chitin-Glucan with a Variable Particle Size

Day creams containing various batches of chitin-glucan with a variable particle size, at the concentration of 1.5%, were prepared according to the same protocol as that of example C1: an unmilled, unscreened batch (L25), and the 3 milled and fractionated batches (L25) of example A2. The ease of formulation and the characteristics of the creams obtained are those in table C2.1.

TABLE C2.1
Characteristics of the creams prepared with the chitin-glucan (L25)
powders with various particle sizes; the sensory aspects (visual aspect
and feel) are classified from 1 to 5, the score 5 corresponding to the
best sensory impressions. The ease of formulation is classified form
0 to 5, the score 1 corresponding to the greatest ease of formulation

| | Size of the chitin-glucan particles | | | | |
|---|---|---|---|---|---|
| | N/A | unmilled, | | 100-200 | 50-90 |
| | (placebo) | unscreened | 200-250 μm | μm | μm |
| Ease of | 1 | 5 | 4 | 2 | 1 |
| formulation | | | | | |
| Sensory: | 3 | 5 | 4 | 3 | 2 |
| visual aspect | | | | | |
| Sensory: feel | 2 | 3 | 2 | 1 | 1 |
| Viscosity | 18000 | 19000 | 19000 | 20000 | 29000 |
| (mPa · s) | | | | | |

It is easily understood from this table that, the finer the size of the particles, the easier it is to incorporate the chitin-glucan into a cosmetic cream and the better the sensory impressions are. It is also seen that the incorporation of the finely milled chitin-glucan, in particular using the fraction 50-90 μm, makes it possible to give the cream considerable viscosity, which is very advantageous for the formulator.

### Example C3

### Influence of the Particle Size of the Chitin-Glucan Powder on the Preparation and the Characteristics of a Lip Balm

A lip balm formulation is prepared with four different particle sizes of the chitin-glucan powder and the ingredients in table C3.1. The powders with a particle size of less than 125, 90, 50 and 30 μm are produced according to the method of example A5, at a concentration of 1.5%. The particle size fraction with a diameter less than 30 μm is obtained after an additional micronization step using an air jet mill.

TABLE C3.1

| | Ingredients | INCI name | % |
|---|---|---|---|
| A | Beeswax | Cera alba | 7 |
| | Carob butter | Octyl dodecanol, | 14.25 |
| | | copernicia cerifera | |
| | | (carnauba) wax | |
| | Coconut oil | Cocos nucifera | 26 |
| | Cocoa butter | Theobroma cacao | 14.25 |
| | Apricot oil | Prunus armeniaca | 15 |
| | Sweet almond oil | Prunus amygdalus dulcis | 12 |
| | Zenigloss UPH | Castor isostearate | 10 |
| | | succinate, hydrogenated | |
| | | castor oil | |
| B | Chitin-glucan; diameter | Chitin, beta-glucan | 1.5 |
| | <125 μm; <90 μm; | | |
| | <50 μm; or <30 μm | | |
| | Natural apricot fragrance | Caprylic/capric triglyceride, | 0.2 |
| | H1504 | prunus armeniaca | |
| | Vanilla extract H1202 | Caprylic/capric triglyceride, | 0.1 |
| | | vanilla planifolia | |
| | Sea-buckthorn oil | Hippophae ramnoides | 0.1 |

Protocol—The ingredients of phase A are mixed at 60° C., and then the mixture is cooled to 45° C. The ingredients of phase B are mixed with phase A and the mixture is stirred for 2-3 minutes. The mixture is immediately introduced into the final packaging, the solidification point being approximately 40° C. The sensory characteristics of the lip balms obtained are those in table C3.2.

TABLE C3.2
Sensory characteristics of the lip balms as a function of the particle size
of the chitin-glucan

| | Diameter of the chitin- | | | |
|---|---|---|---|---|
| | glucan particles | | | |
| | <125 μm | <90 μm | <50 μm | <30 μm |
| Sensory: visual aspect* | 1 | 1 | 2 | 3 |
| Sensory: feel, | 5 | 5 | 5 | 5 |
| sensation on the lips* | | | | |

*The sensory aspects (visual aspect and feel) are classified from 1 to 6, the score 6 corresponding to the best sensory impressions.

It is seen from this example that, in order to be able to obtain a lip balm of professional quality, of homogeneous appearance and with a pleasant feel on the lips, it is necessary to use a chitin-glucan powder of which the diameter is less than 30 μm. With the powders of diameter greater than 30 μm, the lip balm appears to be inhomogeneous, and the chitin-glucan particles are visible and can be felt.

### Example C4

### Preparation of an Antisun Emulsion

An antisun emulsion of “water-in-oil” emulsion type is prepared with the chitin-glucan (1.5%) and the ingredients in table C4.1. Chitin-glucan powders with three different particle sizes prepared according to example A5 were used: <125 μm, <90 μm and <50 μm.

TABLE C4.1

| | Ingredients | INCI name | % |
|---|---|---|---|
| A | Parsol MCX | Ethylhexyl methoxycinnamate | 5 |
| | Parsol 5000 | 4-methyoxydibenzoyl methane | 4 |
| | Parsol SLX | Polysilicone-15 | 3 |
| | Parsol 1789 | Butylmethoxy dibenzoylmethane | 2 |
| | Neoderm AB | C12-C15 alkylbenzoate | 7 |
| | SF1256 | Cyclopentasiloxane, | 4 |
| | | cyclohexasiloxane | |
| | Neoderm IPP | Isopropyl palmitate | 2 |
| | Neoderm ISN | Isononyl isononaoate | 7 |
| | Inutec sp1 | Inulin lauryl carbamate | 0.8 |
| | Sabonal C1618 50/50 | Cetearyl alcohol | 4 |
| | Olive butter | Olea europaea | 1 |
| | Sorbitan stearate | Sorbitan stearate | 0.5 |
| B | Water | Aqua | 22.15 |
| | Euxyl K300 | Phenoxyethanol, methylparaben, | 1 |
| | | ethylparaben, propylparaben, | |
| | | butylparaben | |
| | C1216 | Sucrose laurate | 0.2 |
| | PEG8 | PEG-8 | 2 |
| | 2% xanthan gum | Xanthan gum | 7.5 |
| | Disodium EDTA | Disodium EDTA | 0.05 |
| | Inutec H25P | Inulin | 1 |
| | Veegum HS 5% | Magnesium aluminum silicate | 20 |
| | White Lotus fragrance | Fragrance | 0.3 |
| | UV Titan M170 | Titanium dioxide | 4 |
| | Chitin-glucan <125 μm; | Chitin, beta-glucan | 0 or |
| | <90 μm; <50 μm | | 1.5 |

TABLE C4.2
Characteristics of the antisun emulsions as a function of the particle
size of the chitin-glucan

| | Diameter of the chitin- | | |
|---|---|---|---|
| | glucan particles | | |
| | <125 μm | <90 μm | <50 μm |
| Ease of formulation* | 5 | 5 | 5 |
| Sensory: visual aspect* | 2 | 4 | 4 |
| Sensory: feel* | 4 | 4 | 4 |

*The sensory aspects (visual aspect and feel) are classified from 1 to 5, the score 5 corresponding to the best sensory impressions. The ease of formulation is classified from 0 to 5, the score 5 corresponding to the greatest ease of formulation.

It is seen from this example that, for a formulation of water-in-oil emulsion type like this antisun emulsion, it is preferable to use a chitin-glucan powder with a diameter of less than 90 μm, in order to guarantee a visual aspect that conforms to the manufacturers' requirements.

According to other tests, a copolymer having particles with a diameter of less than 125 μm is suitable for oil-in-water emulsions.

### Example C5

### Preparation of a Tonic Solution

An aqueous tonic solution is prepared with the chitin-glucan (1.5%) and the ingredients in table C5.1. Chitin-glucan powders with two different particle sizes were used: <30 μm and <10 μm. The powders are prepared as in example C3.

TABLE C5.1

| | Ingredients | INCI name | % |
|---|---|---|---|
| A | Isoceteth-20 | Isoceteth-20 | 0.4 |
| | Sensiva-SC50 | Ethylhexylglycerin | 0.1 |
| | Neocare P3C | Polyglyceryl-3 caprate | 0.2 |
| | Euxyl K500 | Potassium sorbate, | 0.6 |
| | | sodium benzoate, | |
| | | imidazoladinyl urea | |
| | MPDIOL | Methylpropanediol | 4 |
| | Water | Aqua | 90.2 |
| | Fucogel 1000PP | Biosaccharide gum-1 | 3 |
| B | Lactic acid | to pH 5.0-6.0 | |
| | Colorant | CI47051 | 0.1 |
| C | Chitin-glucan <30 μm and | Chitin, beta-glucan | 1.5 |
| | <10 μm | | |

Protocol—The ingredients of phase A are mixed in order. Water is added slowly. The pH is adjusted to 5-6 with B, the chitin-glucan is added and mixing is carried out for a few minutes.

TABLE C5.2
Characteristics of the tonic solution as a function of the
particle size of the chitin-glucan

Diameter of the
chitin-glucan
particles

| | <30 μm | <10 μm |
|---|---|---|
| Ease of formulation* | 5 | 5 |
| Sensory: visual aspect* | 1 | 4 |
| Particle stability | 0 | 4 |

*The ease of formulation and the sensory aspects (visual aspect and feel) are classified from 1 to 5, the score 5 corresponding to the best sensory impressions. The ease of formulation is classified from 0 to 5, the score 5 corresponding to the greatest ease of formulation.

It is seen from this example that, in order to prepare a tonic solution with an acceptable appearance and in order for the chitin-glucan particles to remain in a stable suspension, the diameter of the particles must be less than 10 μm.

### Example C6

### Preparation of a Night Cream Containing BDIH-Certifiable Ingredients

A night cream of “water-in-oil” emulsion type is prepared with the chitin-glucan (1.5%) and the ingredients in table C6.1. These ingredients meet all the requirements for certification by the federal association of German commercial and industrial companies that manufacture medicines, dietetic products, food supplements and body care products (BDIH). The cream can obtain the BDIH mark indicating that it has been verified by this association.

Chitin-glucan powders with three different particle sizes prepared according to example C3 were used: <125 μm, <90 μm and <50 μm.

TABLE C6.1

| | Ingredients | INCI name | % |
|---|---|---|---|
| A | Neoderm TCC | Capric/caprylic triglycerides | 11.5 |
| | Neoderm CO | Cetearyl ethylhexanoate | 11.5 |
| | Isolan GTI | Polyglyceryl-4-diisostearate | 4 |
| | Castor wax | Hydrogenated castor oil | 1.5 |
| B | Water | Aqua | Qs |
| | Glycerol | Glycerin | 3 |
| | Magnesium sulfate | Magnesium sulfate | 0.5 |
| | Euxyl K300 | Phenoxyethanol, methylparaben, | 0.8 |
| | | ethylparaben, propylparaben, | |
| | | butylparaben | |
| C | KiOsmetine-CG <125 μm | Chitin, beta-glucan | 1.5 |
| | or <90 μm | | |

TABLE C6.2
Characteristics of the night creams as a function of the particle size of
the chitin-glucan

| | Diameter of the chitin- | | |
|---|---|---|---|
| | glucan particles | | |
| | <125 μm | <90 μm | <50 μm |
| Ease of formulation* | 5 | 5 | 5 |
| Sensory: visual aspect* | 2 | 4 | 4 |
| Sensory: feel* | 2 | 4 | 4 |

*The sensory aspects (visual aspect and feel) are classified from 1 to 5, the score 5 corresponding to the best sensory impressions. The ease of formulation is classified from 0 to 5, the score 5 corresponding to the greatest ease of formulation.

It is seen from this example that, in order to formulate a care product of water-in-oil emulsion type, such as this night cream, it is preferable to use a powder of chitin-glucan with a diameter of less than 90 μm in order to guarantee a visual aspect that complies with the manufacturers' requirements.

### Example C7

### Preparation of Care Formulations of “Water-in-Silicon” Emulsion Type

Two care formulations of “water-in-silicon” emulsion type are prepared with a concentration of chitin-glucan of 1.5% and the ingredients in tables C7.1a and C7.1b.

TABLE C7.1a
Ingredients of formulation 1

| Ingredients | | INCI name | % |
|---|---|---|---|
| A | BY-11-030 | Cyclopentasiloxane, PEG/PPG-19/19 | 8 |
| | | dimethicone | |
| | DC245 | Cyclopentasiloxane | 20 |
| B | Water | Aqua | Qs |
| | NaCl | Sodium chloride | 2 |
| | Preservative | | Qs |
| C | Chitin-glucan <125 μm | Chitin, beta-glucan | 1.5 |
| D | DC9701 | Dimethicone/vinyl dimethicone | 2 |
| | | crosspolymer, silica | |

TABLE C7.2b
Ingredients of formulation 2

| Ingredients | | INCI name | % |
|---|---|---|---|
| A | DC5225 | Cyclopentasiloxane, PEG/PPG-18/18 | 8 |
| | | dimethicone | |
| | DC245 | Cyclopentasiloxane | 20 |
| B | Water | Aqua | Qs |
| | NaCl | Sodium chloride | 2 |
| | Preservative | | Qs |
| C | Chitin-glucan <125 μm | Chitin, beta-glucan | 1.5 |
| D | DC9701 | Dimethicone/vinyl dimethicone | 2 |
| | | crosspolymer, silica | |

Protocol—The ingredients of phase A are mixed at a temperature of 50-60° C. The ingredients of phase B are added to phase A and the mixture is homogenized. C is added with stirring, and the mixture is homogenized until complete incorporation is obtained. D is added and the mixture is homogenized until complete incorporation is obtained.

### Example C8

### Formulation of a Day Care Product

A day care formulation containing 1.5% of chitin-glucan can, for example, be prepared according to the formulation described in C1.

### Example C9

### Formulation of a Body Care Milk

Two body care milk formulations, without chitin-glucan (a) and with chitin-glucan (b), are described below.

| | | | | a | b |
|---|---|---|---|---|---|
| A | Water | Aqua | | 49.5 | 48 |
| | Euxyl K300 | Phenoxyethanol, | Preservative | 1 | 1 |
| | | Methylparaben, | | | |
| | | Ethylparaben, | | | |
| | | Propylparaben, | | | |
| | | Butylparaben, | | | |
| | | Isobutylparaben | | | |
| | Solution | Aqua, | Stabilizer, | 20 | 20 |
| | Carbopol | Acrylates/C10-30 | thickener | | |
| | ETD2020 2% | Alkyl Acrylate | | | |
| | | Crosspolymer | | | |
| | Solution | Aqua, | To minimize | 10 | 10 |
| | Walocel | Hydroxypropyl | rupture on | | |
| | HM400 2% | Methylcellulose | application | | |
| B | Pemulen TR-2 | Acrylates/C10-30 | Stabilizer | 0.3 | 0.3 |
| | | Alkyl Acrylate | | | |
| | | Crosspolymer | | | |
| | Sorbitan laurate | Sorbitan laurate | Co-stabilizer | 0.1 | 0.1 |
| | Argan oil | Argania spinosa | Plant oil | 5 | 5 |
| | Jojoba oil | Simmondsia | Plant wax | 5 | 5 |
| | | chinensis | | | |
| | Neoderm 105 | Isodecyl | | 5 | 5 |
| | | Neopentanoate | | | |
| | Neoderm PTC | Pentaerythrityl | | 2 | 2 |
| | | tetracaprylate, | | | |
| | | Tetracaprate | | | |
| | SF1256 | Cyclopentasiloxane, | Volatile | 2 | 2 |
| | | Cyclohexasiloxane | silicones | | |
| | Fragrance | Fragrance | | 0.1 | 0.1 |
| C | Chitin-glucan | Chitin, Beta-glucan | Restructuring | 0 | 1.5 |
| | (L16, <90 μm) | | agent, firming | | |
| | | | agent | | |
| D | Sodium | Sodium hydroxide | To adjust the | Qs | Qs |
| | hydroxide | | pH to 4.8-5.4 | | |

Protocol—The ingredients of phase A are mixed. The ingredients of phase B are mixed. Phase B is added to phase A, and the mixture is homogenized for 10 minutes at 400 rpm. The powder C is added to the B/A mixture and the mixture is homogenized at 400 rpm for 1 hour. The pH is adjusted to between 4.8 and 5.4 with phase D.

### Example C10

### Formulation of a Firming Cream for the Bust

A firming cream for the bust containing 1.5% of chitin-glucan can, for example, be prepared according to the formulation described below.

| | Ingredients | INCI | Function | % |
|---|---|---|---|---|
| A | Water | Aqua | | 37.55 |
| | EUXYL K300 | Phenoxyethanol, | Preservatives | 0.9 |
| | | Methylparaben, | | |
| | | Butylparaben, | | |
| | | Ethylparaben, | | |
| | | Propylparaben, | | |
| | | Isobutylparaben | | |
| | Glycerol | Glycerin | Stabilizer | 3 |
| | RICE NS | Dimethylimidazolidinone | Feel | 2 |
| | | Rice Starch | | |
| | Veegum | Magnesium aluminum | Stabilizer | 10 |
| | HS 5% | silicate | | |
| | Keltrol T | Xanthan gum | | 0.15 |
| | C1216 | Sucrose laurate | Coemulsifier | 0.2 |
| B | SF1256 | Cyclopentasiloxane, | Volatile | 9 |
| | | Cyclohexasiloxane | silicones | |
| | Shea butter | Butyrospermum Parkii | Treatment | 12 |
| | Olive butter | Olea Eurapaea | Treatment | 10 |
| | Neoderm IPP | Isopropyl Palmitate | Spreading | 6 |
| | Neoderm PTC | Pentaerythrityl | Damper effect | 1.5 |
| | | tetracaprylate/caprate | | |
| | Sabonal C1618 | Cetearyl alcohol | Viscosity | 3 |
| | 50/50 | | modifier | |
| | Neowax FL65 | Glyceryl stearate, PEG-100 | Costabilizer | 2 |
| | | Stearate | | |
| | Rice wax | Oryza Sativa | Hardness | 0.2 |
| | Inutec SP1 | Inulin Lauryl Carbamate | Stabilizer | 0.8 |
| C | Ginger Lychee | Fragrance | | 0.5 |
| | 506340 | | | |
| | Chitin-glucan | Chitin, Beta-glucan | Firming agent | 1.5 |
| | (L16, fraction | | | |
| | <90 μm) | | | |
| D | Caroblend | 99.95% (PPG-15 Stearyl | Colorant | 0.2 |
| | (0.05% | Ether), 0.05% | | |
| | Caroquest | (Carotenoids, | | |
| | MCT) | Caprylic/Capric | | |
| | | Triglyceride) | | |

Procedure—The ingredients of phase A are mixed at 80° C., and then the ingredients of phase B are mixed at 85° C. Phase B is added to phase A, and the mixture is homogenized for 5 minutes at 10 000 rpm and then left to cool to 40° C. The ingredients of phases C and D are added to the emulsion with stirring.

### Example C11

### Formulation of a Baby Care Milk

A baby care milk containing 1.5% of chitin-glucan can, for example, be prepared according to the formulation described below.

| | Ingredients | INCI | Function | % |
|---|---|---|---|---|
| A | Shea butter | Butyrospermum parkii | Plant butter | 15 |
| | Olive butter | Olea europaea | Plant butter | 10 |
| | Zenebicream | Octyl dodecanol, | Emollient | 5 |
| | | Beeswax | | |
| | Sweet almond | Prunus | Plant oil | 15 |
| | oil | amygdalus | | |
| | | dulcis | | |
| | Neoderm PTC | Pentaerythrityl | Texturing agent | 4 |
| | | tetracaprylate, | | |
| | | Tetracaprate | | |
| | Vaseline | Petroleum jelly | Occlusive wax | 1 |
| | Sabonal C1618 | Cetearyl alcohol | Stabilizer, | 2 |
| | 50/50 | | sensory | |
| | | | perception | |
| | Sorbitan stearate | Sorbitan Stearate | Emulsifier | 2 |
| | Inutec SP1 | Inulin lauryl carbamate | Stabilizes the | 1 |
| | | | emulsion | |
| | Zinc oxide | Zinc oxide | Anti-irritant | 4 |
| B | Water | Aqua | | 11.35 |
| | Keltrol T | Xanthan Gum | Thickener | 0.05 |
| | Neocare SC15 | Sucrose cocoate | Coemulsifier | 0.4 |
| | Corn PO4 | Distarch phosphate | Moisturizer | 4 |
| C | Inutec H25 | Inulin | Conditions | 23.5 |
| D | Fragrance | Perfume | the skin | 0.2 |
| E | Chitin-glucan | (Chitin, Beta-glucan | Reinforces | 1.5 |
| | (L16, fraction | | the barrier | |
| | <90 μm) | | effect of the skin, | |
| | | | protects the skin | |

Procedure—The ingredients of phase A are mixed at 75° C., and then the ingredients of phase B are mixed at 80° C. Phase A is added to phase B, and the mixture is homogenized for 3 minutes at 10 000 rpm, and then left to cool to 40° C. The ingredients of phases C and D are added to the emulsion with stirring, and the mixture is then homogenized for 1 minute at 10 000 rpm. The chitin-glucan powder (E) is added and the whole is stirred for 60 minutes (for a final amount of 200 g).

### Example C12

### Formulation of a Hand Cream

A hand cream containing 1.5% of chitin-glucan can, for example, be prepared according to the formulation described below.

| | Ingredients | INCI | Function | % |
|---|---|---|---|---|
| A | Water | Aqua | | Qs |
| | Glycerol | Glycerin | Stability under | 3 |
| | | | cold conditions | |
| | Euxyl K300 | Phenoxyethanol, | Preservative | 0.8 |
| | | Ethylparaben, | | |
| | | Methylparaben, | | |
| | | Propylparaben, | | |
| | | Butylparaben, | | |
| | | Isobutylparaben | | |
| | Keltrol T | Xanthan gum | Anticreaming | 0.1 |
| | Neocare Olive | Olive oil hydrolyzed wheat | Emulsifier | 4 |
| | | protein, Cetearyl alcohol, | | |
| | | Glyceryl oleate, Glyceryl | | |
| | | stearate, Potassium | | |
| | | hydroxide | | |
| | Inutec SP1 | Inulin lauryl carbamate | Costabilizer | 0.3 |
| | Dimethicone | Dimethicone | Anti-whitening | 5 |
| | M350 | | | |
| B | Shea butter | Butyrospermum parkii | Plant butter | 3 |
| | SF1214 | Cyclopentasiloxane, | Silicone resin | 1 |
| | | Dimethicone | | |
| | Neoderm PTC | Pentaerythrityl | Damper effect | 2 |
| | | capric/caprylate | | |
| | Neoderm TCC | Capric/caprylic triglyceride | Neutral oil | 3 |
| | Apricot oil | Prunus armeniaca | Plant oil | 3 |
| | Sabonal C1618 | Cetearyl alcohol | Stabilizer | 3 |
| | 50/50 | | | |
| | Chitin-glucan | Chitin, Beta-glucan | Protecting and | 1.5 |
| | (L16, fraction | | regenerating | |
| | <90 μm) | | agent | |
| C | Panthenol | Panthenol | Regenerating | 1 |
| | | | agent | |
| | Fragrance | Perfume | Fragrance | 0.2 |

Protocol—The ingredients of phase A are mixed at 65° C., and then the ingredients of phase B are mixed at 65° C. Phase A is added to phase B, and the mixture is homogenized for 3 minutes at 10 000 rpm, and then left to cool to 40° C. The ingredients C are added to the emulsion, and the mixture is cooled, and then mixed for at least 30 minutes.

### Example C13

### Formulation of an Anti-Acne Lotion

An anti-acne lotion containing 1.5% of chitin-glucan can, for example, be prepared according to the formulation described below.

| | Ingredients | INCI | Function | % |
|---|---|---|---|---|
| A | Neoderm ISN | Isononyl isononanoate | Sensory | 5 |
| | | | perception | |
| | Neoderm 105 | Isodecyl neopentanoate | Improves | 5 |
| | | | spreading | |
| | Neoderm AB | C12-15 Alkyl benzoate | Dry feel | 5 |
| | Jojoba oil | Simmondsia chinensis | Plant oil | 1 |
| | Inutec SP1 | Inulin lauryl carbamate | Stabilizes the | 0.2 |
| | | | emulsion | |
| | Tea tree oil | Melaleuca Alternifolia | Essential oil | 2 |
| | Pemulen TR2 | Acrylates/C10-30 Alkyl | Emulsifier | 0.3 |
| | | Acrylate Crosspolymer | | |
| B | Water | Aqua | | 46.1 |
| | Euxyl K300 | Phenoxyethanol, | Preservative | 0.9 |
| | | Methylparaben, | | |
| | | Butylparaben, | | |
| | | Ethylparaben, | | |
| | | Propylparaben, | | |
| | | Isobutylparaben | | |
| | Carbopol EDT | Acrylates/C10-30 Alkyl | Thickener | 20 |
| | 2020 2% | Acrylate Crosspolymer | | |
| | Walocel HM4000 | Hydroxypropyl | Thickener | 10 |
| | 2% | methylcellulose | | |
| | Chitin-glucan | Chitin, Beta-glucan | Purifying agent | 1.5 |
| | (L16, | | | |
| | fraction <90 μm) | | | |
| | Corn PO4 | Distarch phosphate | Moisturizer | 3 |

### Procedure

The ingredients of phase A are mixed. The ingredients of phase B are mixed. Phase A is added to phase B, and the mixture is homogenized for 3 minutes at 10 000 rpm (for a final amount of 200 g).

### Example C14

### Formulation of an Anti-Psoriasis Care Product

An anti-psoriasis care product containing 1.5% of chitin-glucan can, for example, be prepared according to the formulation described below.

| | Ingredients | INCI | Function | % |
|---|---|---|---|---|
| A | Water | Aqua | | 38.2 |
| | Avicel PC 591 | Microcrystalline | Thickener, stabilizer | 1 |
| | | Cellulose, Cellulose | | |
| | | Gum | | |
| | Chitin-glucan | Chitin, Beta-glucan | Anti-psoriasis active | 1.5 |
| | (L16, fraction | | agent | |
| | <90 μm) | | | |
| B | Neocare P3C | Polyglyceryl-3 | Anti-bacterial | 1 |
| | | Caprylate | solvent | |
| | Centapowder | Centella asiatica | Regulates cell | 0.1 |
| | | | proliferation | |
| | Fragrance | Perfume | | 0.2 |
| C | Borage oil | Borago officinalis | Plant oil | 30 |
| | Rose oil | Rosa moschata | Plant oil | 20 |
| | Macadamia oil | Macadamia ternifolia | Plant oil | 5 |
| | Sabonal C1618 | Cetearyl alcohol | Stabilizer, sensory | 2 |
| | 50/50 | | perception | |
| | Inutec SP1 | Inulin lauryl | Stabilizes the | 1.2 |
| | | carbamate | emulsion | |

Procedure—The ingredients of phase A are mixed. The ingredients of phase C are mixed, and phase C is brought to 75° C. Phase C is added to phase A with, stirring, and the mixture is homogenized for 3 minutes at 10 000 rpm, and then cooled to 40° C. The ingredients of phase B are mixed, and then phase B is added to the C/A emulsion. The mixture is homogenized for 1 minute at 10 000 rpm (for a final amount of 400 g).

### Series of Examples ‘D’. Effects of the Regular Application of Chitin-Glucan-Based Cosmetic Creams on the Characteristics of the Skin

### Example D1

### Study of the Effects of the Daily Application of a Day Cream Based on Chitin-Glucan at a Concentration of 1.5%, for 4 Months, on the Biometric Parameters of the skin of Elderly Individuals

This example illustrates the effects of the cream described in example C1, which contains 1.5% of the chitin-glucan (reference T1.5), on various characteristics of the skin, in particular in the context of skin aging, the cream and its placebo being applied to the forearm of elderly individuals for a period of 4 months. Since the placebo cream is a formulation that is itself very hydrating, the study clearly demonstrates the effects of the milled chitin-glucan.

Protocol—The study was carried out on 20 male volunteers, 58±4 years old, who applied the chitin-glucan-based cream (T1.5) and the placebo cream blind, each on one forearm, at a rate of twice a day for 4 months. Five types of clinical and biometrological examinations were carried out monthly. At each evaluation time, the interproduct comparison was carried out by means of Friedman's non-parametric test followed by Dunn's test. For each product, the comparison over time was carried out according to the same methods. For each parameter, significant differences between the values obtained at the various times were sought by means of Friedman's non-parametric paired test followed by Dunn's test (value p).

Results—

1—Evaluation of the structure of the surface of rough skin by squamometry X—Squamometry X consists in taking a sample from the surface of the horny layer by means of a transparent self-adhesive disk applied for about ten seconds under a pressure of 110 g/cm² provided by a dynamometer. The horny layer sample is stained with a solution of toluidine blue and basic fuchsin. The color defined by the Chroma C* measured by reflectance colorimetry (Minolta Chroma meter) evaluates the state of xerosis. “Normal” skin, which is smooth and well hydrated, has a squamometry index C* of approximately 5 to 7. The higher the value, the thicker the horny layer, and the rougher and drier the skin. At the start of the study (M0), the two sites that were to receive the test formulations had equivalent squamometry index values. The Chroma C* values subsequently obtained are those in table D1.1. For the formulation T0, a significant decrease was observed after one month (M1) of treatment (p<0.05), and the subsequent months (p<0.001). For the formulation T1.5, the improvement was very significant and was present from the second month (M2) of treatment onward (p<0.001). This was also the case between the first and the third month (M3) (p<0.01), and the first and the fourth month (M4) (p<0.001). The comparison between the effectiveness of the two formulations revealed that T1.5 was clearly superior from the end of the first month of treatment and during the subsequent months (p<0.001).

TABLE D1.1
Squamometry index (Chroma C*) indicating the squamous state of the
skin

| Time | C* | C* | Inter-product |
|---|---|---|---|
| (months) | Cream T1.5 | Placebo T0 | comparison |
| M0 | 11.95 ± 2.26 | 11.82 ± 2.27 | NS |
| M1 | 8.79 ± 2.3 | 10.49 ± 2.59 | p < 0.001 |
| M2 | 7.3 ± 2.6 | 9.81 ± 3.56 | p < 0.0001 |
| M3 | 6.62 ± 2.2 | 9.54 ± 3.21 | p < 0.0001 |
| M4 | 5.8 ± 1.8 | 9.22 ± 3.46 | p < 0.0001 |

Inter-time comparison (p)

| | M1 | M2 | M3 | M4 |
|---|---|---|---|---|
| Placebo T0 | | | | |
| M0 | p < 0.05 | p < 0.001 | p < 0.001 | p < 0.001 |
| M1 | | NS | NS | NS |
| M2 | | | NS | NS |
| M3 | | | | NS |
| Cream T1.5 | | | | |
| M0 | NS | p < 0.001 | p < 0.001 | p < 0.001 |
| M1 | | NS | p < 0.01 | p < 0.001 |
| M2 | | | NS | NS |
| M3 | | | | NS |

NS: values not significantly different;
M0 signifying 0 months, etc.

2. Evaluation of the heterogeneity of the skin color by ultraviolet light enhanced visualization (ULEV)—The ULEV method (Visioscan) is a noninvasive method which demonstrates the squamous state of the skin, in particular the fine squamae undergoing detachment, and characterizes the cohesion of the corneocytes of the horny layer. The measurement is expressed as percentage of the skin surface affected by the process. When the skin is smooth and the horny layer is cohesive, the ULEV percentage is low, of the order of 5-6%.

At the time of inclusion in the study (M0), and after one month of treatment (M1), the percentage of the skin surface affected by the desquamation process was similar on the two sites treated, approximately 8-9%. The values subsequently obtained are those in table D1.2. The placebo formulation T0 provided a significant improvement (p<0.001) from the first month, and said improvement persisted for the rest of the study and reached a value of 7.4% at 4 months. The formulation T1.5 also provided a significant improvement, which could be seen from the first month (p<0.01) and became stronger during the following months (p<0.001). This could be seen through improvements between the first and the fourth months (p<0.01) and between the second and the fourth months (p<0.05). The comparison between the two sites treated demonstrates the superiority of the formulation T1.5 at the second month (p<0.01) and also at the third and fourth months (p<0.001) compared to the placebo cream.

TABLE D1.2
Percentage of the skin surface affected by the desquamation process
(measured by ULEV “ultraviolet light enhanced visualization”, Visioscan)

| Time | % | % | Inter-product |
|---|---|---|---|
| (months) | Cream T1.5 | Placebo T0 | comparison |
| M0 | 8.85 ± 2.29 | 8.82 ± 2.26 | NS |
| M1 | 7.95 ± 2.04 | 8.57 ± 2.04 | p < 0.01 |
| M2 | 6.23 ± 2.18 | 7.83 ± 2.67 | p < 0.001 |
| M3 | 6.06 ± 2.12 | 8.24 ± 2.88 | p < 0.001 |
| M4 | 5.11 ± 1.6 | 7.38 ± 2.03 | p < 0.0001 |

Inter-time comparison (p)

| | M1 | M2 | M3 | M4 |
|---|---|---|---|---|
| Placebo T0 | | | | |
| M0 | p < 0.001 | p < 0.001 | p < 0.001 | p < 0.001 |
| M1 | | NS | NS | NS |
| M2 | | | NS | NS |
| M3 | | | | NS |
| Cream T1.5 | | | | |
| M0 | p < 0.01 | p < 0.001 | p < 0.001 | p < 0.001 |
| M1 | | NS | NS | p < 0.01 |
| M2 | | | NS | p < 0.05 |
| M3 | | | | NS |

NS: values not significantly different;
M0 signifying 0 months, etc.

3. Evaluation of the relief of the skin by optical profilometry—The relief of the skin was observed noninvasively by optical profilometry (Hitachi camera), and the prolifometry graphs were analyzed in order to calculate the average depth of the roughness of the skin, Rz (μm).

At the time of inclusion in the study (M0), no difference in the Rz value was present between the two sites. The average depth values subsequently obtained are those in table D1.3. The formulation T0 significantly reduces (p<0.01) the Rz value at the end of the fourth month of treatment. The formulation T1.5 reduces the Rz value highly significantly (p<0.001) from the second month of treatment, until the end of the study. The improvement was also marked between the first and third month of treatment (p<0.05) and also between the first and the fourth month (p<0.001) and the second and the fourth month (p<0.05). Compared with the placebo cream, the formulation T1.5 was found to be the more effective from the first month (p<0.01), and also the subsequent months (p<0.001).

FIG. 5 shows the graphs of profilometry obtained on the two forearms of an individual after 16 weeks of application of the T1.5 cream and of the T0 cream.

TABLE D1.3
Average depth of the roughness of the skin Rz (μm),
by optical profilometry

| Time | Rz (μm) | Rz (μm) | Inter-product |
|---|---|---|---|
| (months) | Cream T1.5 | Placebo T0 | comparison |
| M0 | 8.85 ± 2.29 | 8.82 ± 2.26 | NS |
| M1 | 7.95 ± 2.04 | 8.57 ± 2.04 | p < 0.01 |
| M2 | 6.23 ± 2.18 | 7.83 ± 2.67 | p < 0.001 |
| M3 | 6.06 ± 2.12 | 8.24 ± 2.88 | p < 0.001 |
| M4 | 5.11 ± 1.6 | 7.38 ± 2.03 | p < 0.0001 |

Inter-time comparison (p)

| | M1 | M2 | M3 | M4 |
|---|---|---|---|---|
| Placebo T0 | | | | |
| M0 | NS | NS | NS | p < 0.01 |
| M1 | | NS | NS | NS |
| M2 | | | NS | NS |
| M3 | | | | NS |
| Cream T1.5 | | | | |
| M0 | NS | p < 0.001 | p < 0.001 | p < 0.001 |
| M1 | | NS | p < 0.05 | p < 0.001 |
| M2 | | | NS | p < 0.05 |
| M3 | | | | NS |

NS: values not significantly different;
M0 signifying 0 months, etc.

4. Evaluation of the tonicity/elasticity of the skin by measuring the resonance running time in the skin—The resonance running time measurement in the skin (RRTM, arbitrary value) is measured between two strips affixed on the skin, by a Reviscometer. The RRTM is a good indicator of the intrinsic tension and of the tonicity of the skin, since the more tonic and taut the skin is, the less it forms raised folds, and more rapidly the ultrasound wave propagates. Thus, the shorter the RRTM, the more taut are the parts constituting the skin. The RRTM is also influenced by the state of the horny layer, its cohesion; the more normalized the horny layer, the shorter the RRTM.

The RRTM values are similar between the two sites at the beginning of the study and after one month of the trial. The values subsequently obtained are those in table D1.4. The formulation T0 did not bring about any modification of the RRTM value in the course of the treatment. The formulation T1.5 induced a significant reduction in RRTM after three months (p<0.01) and four months (p<0.001), and also between the first and, respectively, the second month (p<0.01) and the third and four months (p<0.001). Compared with the placebo formula, the cream T1.5 was found to be significantly more effective after two and three months (p<0.01), and also after four months (p<0.001).

TABLE D1.4
Resonance running time measurement
(RRTM arbitrary unit, Reviscometer)

| Time | | | Inter-product |
|---|---|---|---|
| (months) | Cream T1.5 | Placebo T0 | comparison |
| M0 | 326 ± 64 | 327 ± 61 | NS |
| M1 | 329 ± 58 | 329 ± 54 | NS |
| M2 | 306 ± 68 | 329 ± 60 | p < 0.01 |
| M3 | 302 ± 60 | 322 ± 52 | p < 0.01 |
| M4 | 291 ± 59 | 322 ± 56 | p < 0.0001 |

Inter-time comparison (p)

| | M1 | M2 | M3 | M4 |
|---|---|---|---|---|
| placebo T0 | | | | |
| M0 | NS | NS | NS | NS |
| M1 | | NS | NS | NS |
| M2 | | | NS | NS |
| M3 | | | | NS |
| cream T1.5 | | | | |
| M0 | NS | NS | p < 0.01 | p < 0.001 |
| M1 | | p < 0.01 | p < 0.001 | p < 0.001 |
| M2 | | | NS | NS |
| M3 | | | | NS |

NS: values not significantly different;
M0 signifying 0 months, etc.

5. Evaluation of the hydration of the horny layer by measuring capacitance—The hydration of the horny layer is evaluated by measuring electrical capacitance using a Corneometer (arbitrary value). The capacitance of the skin is proportional to the water content of the skin and to the hydration state of the corneocytes of the horny layer. Well hydrated skin has a capacitance of 65, very dry skin of 30 to 40.

The capacitance values are similar on the two sites at the start of the study. The values subsequently obtained are those in table D1.5. The placebo formulation T0 made it possible to increase the capacitance significantly after one month (p<0.05) and after four months (p<0.001). In the intermediate times of two and three months, no effect of the application of the creams is observed on the capacitance measurement. The formulation T1.5 allows a significant increase in the capacitance after one month (p<0.05), and also after 2 and 4 months (p<0.001). A significant increase (p<0.05) in the capacitance is also observed between the first and the fourth months. The cream T1.5 is significantly more effective than the placebo cream after one month (p<0.05), and even more significantly effective at 2 and 4 months (p<0.001).

TABLE D1.5
Hydration of the horny layer by determination of the electrical
capacitance of the skin (Corneometer)

| Time | | | Inter-product |
|---|---|---|---|
| (months) | Cream T1.5 | Placebo T0 | comparison |
| M0 | 57.7 ± 4.0 | 54.8 ± 3.1 | n |
| M1 | 61.3 ± 3.0 | 57.3 ± 13.1 | p < 0.05 |
| M2 | 62.9 ± 2.7 | 57.2 ± 12.9 | p < 0.001 |
| M3 | 63.5 ± 1.8 | 57.2 ± 12.6 | p < 0.001 |
| M4 | 64.6 ± 1.9 | 58.0 ± 12.5 | p < 0.001 |

Inter-time comparison (p)

| | 1 | 2 | 3 | 4 |
|---|---|---|---|---|
| Placebo T0 | | | | |
| M0 | p < 0.05 | NS | NS | p < 0.001 |
| M1 | | NS | NS | NS |
| M2 | | | NS | NS |
| M3 | | | | NS |
| Cream T1.5 | | | | |
| M0 | p < 0.05 | p < 0.001 | p < 0.001 | p < 0.001 |
| M1 | | NS | NS | p < 0.05 |
| M2 | | | NS | NS |
| M3 | | | | NS |

NS: values not significantly different;
M0 signifying 0 months, etc.

Conclusions—The five biometrological parameters all indicate the superiority of the chitin-glucan-based cream (T1.5) compared with the placebo cream (T0) containing the same ingredients except the chitin-glucan. Elderly skin becomes smoother, less squamous, more hydrated, firmer and less xerotic. The results indicate that the chitin-glucan-based cream exercises a set of actions that allows the skin to regain its elasticity, and thereby appear smoother, with less relief, and more radiant, making it possible to describe it as an anti-aging ingredient. The results are partly explained by a considerable and sustained hydrating effect, as indicated by the corneometry and capacitance tests, but other deeper and long-lasting mechanisms of action are also involved, allowing the skin to restore its barrier function and to have its metabolic and defensive activities stimulated.

### Example D2

### Study of the Effects of Daily Application of a Day Cream Based on Chitin-Glucan at a Concentration of 1.5%, for 4 Months, on the Wrinkles and the Surface Topology of the skin in the Region of the Eyes of Elderly Individuals

This example illustrates the effects of a cream based on 1.5% of chitin-glucan on various characteristics of the skin, in particular in the context of skin aging, the test cream and its placebo being applied to the area of the corner of the eye of elderly individuals, daily, for a period of 4 months. The day cream is an emulsion of oil-in-water type prepared with the ingredients in table D2.1, with chitin-glucan with a particle size of less than 125 μm produced according to example A5, and without chitin-glucan for the placebo cream.

TABLE D2.1
Day cream with chitin-glucan with a particle size <125 μm (test) and
without chitin-glucan (placebo)

| | Ingredients | INCI name | % |
|---|---|---|---|
| A | Water | Aqua | 20.1 |
| | Glycerol | Glycerin | 3 |
| | EULYPE 9010 | Phenoxyethanol, | 1 |
| | | ethylhexylglycerin | |
| | Cellolize QP5200 2% | Hydroxyethylcellulose | 25 |
| | 2% xanthan gum | Xanthan gum | 20 |
| | Neocare SC15 | Sucrose cocoate | 0.6 |
| | Corn P04 | Distarch phosphate | 3 |
| B | Sorbitan stearate | Sorbitan laurate | 4.5 |
| | Inutec SP1 | Inulin lauryl carbamate | 0.2 |
| | Neoderm TCC | Capric/caprylic triglyceride | 8 |
| | Neoderm PTC | Pentaerythrityl tetracaprylate/ | 2 |
| | | caprate | |
| | Apricot oil | Prunus armeniaca | 5 |
| | Jojoba butter | Butyrospermum parkii | 2 |
| | Parsol 1789 | Butylmethoxy dibenzoylmethane | 1 |
| | Sabonal C 1618 50/50 | Cetearyl alcohol | 3 |
| C | Fragrance | Perfume | 0.1 |
| | Lactic acid | Lactic acid | |
| D | Chitin-glucan <125 μm | Chitin, beta-glucan | 0 or 1.5 |

Protocol—The single-blind, intra-individual study was carried out on 21 female volunteers, 59±1 years old (between 52 and 65 years old) having deep wrinkles in the crows feet, who applied the chitin-glucan-based cream (test cream) and the cream without chitin-glucan (placebo) to the skin in the region of the corner of the eye (crows foot), at a rate of twice a day for 4 months. At the beginning of the study and then after 1, 2, 3 and 4 months of use, the skin relief parameters were characterized using photographs of the imprint of the skin in a SilFlo® gel analyzed using the Skin Image Analyser®. The photographs are taken at an angle of 35° so as to allow visualization of the shadow areas. The Quantirides® image analysis software gives the total wrinkle surface area, all the wrinkles having a minimum surface area of 0.03 mm² being detected. It thus gives the number and the average depth of the wrinkles, in particular of the wrinkles of the microrelief of the skin. The variation in the parameters is subsequently calculated by comparing:
The mean of the parameter for all of the volunteers at time t with the mean of the parameter at time zero (Δ1)
The mean of the parameter for all the volunteers at a time t for the area treated with the test cream with chitin-glucan, with the mean of the parameter for the area treated with the placebo cream (Δ2)
   According to the following formulae:

$$Δ 1 ( \% ) = \frac{{X}_{t} - {X}_{t 0}}{{X}_{t 0}} \cdot 100$$

$$Δ 2 = ( {X}_{Tt} - {X}_{Tt 0} ) - ( {X}_{Pt} - {X}_{Pt 0} )$$

$$and$$

$$Δ 2 ( \% ) = \frac{Δ 2}{{X}_{Tt 0} + ( {X}_{P t} - {X}_{Pt 0} )} \cdot 100$$

### Where:

Xₜ and Xₜ₀ are the mean values of the parameter X, obtained with the test cream or the placebo at times t and t0;
X_{Tt} and X_{Tt0} are the mean values of the parameter X, obtained with the test cream (with chitin-glucan) at times t and t0;
X_{Pt} and X_{Pt0} are the mean values of the parameter X, obtained with the placebo cream (without chitin-glucan) at times t and t0.

Statistics—Significant differences between two parameters (time t versus time zero, or test cream versus placebo cream) were sought by means of Friedman's non-parametric paired test followed by Dunn's test.

Results—

TABLE D2.2
Variation in the mean of the parameters of the skin relief at
time t compared with time zero for the cream with chitin-glucan
(test) and without chitin-glucan (placebo)

| | t | Δ1 (%) | | Δ1 (%) | |
|---|---|---|---|---|---|
| | (months) | test | p | placebo | p |
| Total wrinkled | M1 | −2 ± 1.3 | NS | 1 ± 1.5 | NS |
| surface area (mm²) | M3 | −5.6 ± 1.5 | p < 0.05 | 0.4 ± 1.4 | NS |
| | M4 | −4.1 ± 1.7 | p < 0.05 | 0.3 ± 1.3 | NS |
| Number of folds of | M1 | 1 ± 2 | NS | 4 ± 3 | NS |
| the microrelief | M3 | 1 ± 2 | NS | 4 ± 3 | NS |
| | M4 | −3 ± 1 | p < 0.05 | 3 ± 3 | NS |
| Depth of the folds | M1 | −0.5 ± 0.6 | NS | 1.2 ± 0.8 | NS |
| of the microrelief | M3 | −0.7 ± 2 | NS | 0.4 ± 0.7 | NS |
| (μm) | M4 | −0.9 ± 0.4 | p < 0.05 | 0.7 ± 0.5 | NS |

NS: difference not significant

TABLE D2.3
Variation in the mean of the parameters of the skin relief for all
the volunteers at a time t for the area treated with the test cream
with chitin-glucan, compared with the variation in the mean of the
parameter for the area treated with the placebo cream

| | t | | |
|---|---|---|---|
| | (months) | Δ2 (%) | p |
| Total wrinkled | M1 | −3.2 ± 2.1 | NS |
| surface area (mm²) | M3 | −3 ± 1.3 | p < 0.05 |
| | M4 | −4.5 ± 2.1 | p < 0.05 |
| Number of folds of | M1 | −2 ± 3 | NS |
| the microrelief | M3 | −2 ± 3 | NS |
| | M4 | −5 ± 2 | p < 0.05 |
| Depth of the folds of | M1 | −1.3 ± 1 | NS |
| the microrelief (μm) | M3 | −1.4 ± 1 | NS |
| | M4 | −1.6 ± 0.7 | p < 0.05 |

It is deduced from this example that a cream of oil-in-water emulsion type containing 1.5% of chitin-glucan with a particle size of less than 125 μm makes it possible to smooth out the surface of the skin in the crows foot region and to visibly reduce the number and the depth of the wrinkles, in particular of the folds of the microrelief. The relief of the skin is characterized by:
a total wrinkled surface area which is significantly lower from 3 months of use of the chitin-glucan-based cream onward, both in comparison with time zero (A1) and in comparison with the use of the placebo cream (Δ2);
a depth and a number of folds of the microrelief significantly lower after 4 months of use of the chitin-glucan-based cream, both in comparison with time zero (A1) and in comparison with the use of the placebo cream (Δ2)

### Example ‘D3’

### Anti-Aging Effect of a Composition Containing Chitin-Glucan with a Fine and Controlled Article Size. Study of the Production of Procollagen I in a Model of Coculture of Reconstructed Human Epidermis and of Human Fibroblasts in the Presence of Chitin-Glucan

The biological model used comprises a reconstructed human epidermis (0.5 cm², 5 days) and fibroblasts originating from normal human epidermis (PF2, eighth passage) placed in a 24-well plate (one reconstructed epidermis and 120 000 fibroblasts per well), cultured in a DMEM/HAM F12 supplemented cocultured medium.

A simple formulation of oil-in-water emulsion type prepared with the ingredients in table D3.1, with a chitin-glucan (batch L25) with a particle size of less than 125 μm, is applied topically to the epidermis (5 mg/cm²). Two studies are carried out: a cytotoxicity study (cell viability of the epidermis and of the fibroblasts), and a study of procollagen I production in the culture medium.

TABLE D3.1

| | Ingredients | INCI name | % |
|---|---|---|---|
| A | Water | Aqua | Qs |
| | Glycerol | Glycerin | 35% |
| | Lactic acid | Qs (to pH 4.5) | Qs |
| B | Neoderm TCC | Capric/caprylic triglyceride | 5 |
| | Simulgel EPG | Sodium acrylate/acryloyldimethyl | 2.8 |
| | | taurate copolymer, polyisobutene, | |
| | | caprylylcapryl glucoside | |
| C | Chitin-glucan <125 μm; | Chitin, beta-glucan | 1.5 |
| | <90 μm; <50 μm | | |

Cytotoxicity—After topical application of the test cream, the epidermis in coculture with the fibroblasts are incubated at 37° C. with 5% CO₂ for 48 hours. The cell viability of the fibroblasts and of the epidermis is estimated by calorimetric labeling of the live cells with MTT (3-(4,5-dimethylthiazol-2-yl)-2,5-diphenyltetrazolium bromide), and also by visual evaluation of the morphology of the fibroblast cells. The cell viability of the cultures treated with the test cream is compared with that of the untreated cultures (control). Three cultures per treatment type are carried out. The results are those in table D3.2.

TABLE D3.2
Effect of the topical application of a cream containing 1.5% of chitin-
glucan on the viability of a reconstructed epidermis in a fibroblast
coculture, and of the fibroblasts, after a contact time of 48 hours

| | Viability of | Viability of the |
|---|---|---|
| | the epidermis | fibroblasts |
| Topical treatment | (%) | (%) |
| None (control) | 100 | 100 |
| Test cream with 1.5% chitin- | 94 | 100 |
| glucan | | |

Production of procollagen I—The epidermis/fibroblast cocultures are placed under 3 different conditions:
topical application of the test cream with 1.5% chitin-glucan;
no treatment (control);
addition of a solution of TGF-β and of ascorbic acid in order to supplement the culture medium and to maximize the collagen production by the fibroblasts (reference).
   3 cocultures per condition are incubated at 37° C. with 5% CO₂ for 48 hours. The concentration of procollagen I in solution in the medium is determined by means of an ELISA assay (procollagen type I C-peptide EIA kit, BioWhittaker MK101). The comparison between the results of the various groups is calculated by analysis of variance (ANOVA) with Dunnett's multiple comparison test (Table D3.3).

TABLE D3.3
Effect of the topical application of a chitin-glucan-based cream on the
production of procollagen type I in the epidermis/fibroblast coculture
media (n = 3): parameter p for the comparison with the control group

| | Concentration | |
|---|---|---|
| | of procollagen I | |
| Treatment | (ng/ml) | p |
| Control (none) | 3458 ± 252 (100%) | — |
| Topical application of the cream with | 6693 ± 412 (194%) | p < 0.01 |
| 1.5% chitin-glucan | | |
| Supplementation of the culture | 9980 ± 571 (289%) | p < 0.01 |
| medium with TGF-β/ascorbic acid | | |
| (reference) | | |

It is learnt from this example that the topical application of a cream based on chitin-glucan with a particle size of less than 125 μm at the concentration of 1.5%, to a reconstructed epidermis in coculture with fibroblasts, is nontoxic for the epidermis and the fibroblasts, and that it very significantly promotes the production of procollagen type I in the culture medium (secreted by the cells of the epidermis and/or the fibroblasts) compared with the control group without treatment. The cream based on chitin-glucan with a particle size of less than 125 μm at the concentration of 1.5% therefore exercises an anti-aging action on the skin, procollagen type I being the precursor of collagen, the main component of the extracellular matrix of the dermis.

### Example ‘D4’

### Anti-Aging Effect of a Composition Containing Chitin-Glucan with a Fine and Controlled Article Size. Evaluation of the Protective Effects of the Chitin-Glucan on Langerhans Cells in Human Skin Biopsies Exposed to UVB Radiation

Protocol—Langerhans cells are dendritic cells located predominantly in the deepest layers of the epidermis. They do not contain melanin and are very sensitive to external attacks such as exposure to UV radiation. In the event of external stress, they have a tendency to migrate from the epidermis to the dermis, and then to trigger the activation of lymphocytes. The number of healthy Langerhans cells present in the epidermis is therefore used as an indicator of stress-related and aging-related skin damage. The model used is a skin explant originating from a biopsy (4 cm²), cultured on a 6-well plate in a culture medium (DMEM, 2 mM L-glutamine, 50 IU/ml-50 μg/ml penicillin-streptomycin), 10% fetal calf serum at 37° C. (95% air and 5% CO2). The effects of the following 3 treatments are compared:
control without topical treatment;
topical application of 5 mg/cm² of a cream containing 1.5% of chitin-glucan with a particle size of less than 125 μm, prepared according to example 29;
topical application of 5 mg/cm² of an antisun cream with a protection factor of 20 (reference).
   Two studies are carried out: a cytotoxicity study, and a study of the effect of the chitin-glucan-based cream on the number of Langerhans cells in the explants which have or have not been irradiated with UVB radiation.
   Cytotoxicity—It is characterized by the cell viability of the cells of the explant (visualization by means of the MTT test as in example 29), 24 hours after treatment. The results are those in table D4.1.

TABLE D4.1
Effect of the topical application of a cream containing 1.5% of chitin-
glucan on the viability of a skin explant after a contact time
of 48 hours on the epidermis

| | Viability of the |
|---|---|
| Topical treatment | epidermis (%) |
| None (control) | 100 |
| Test cream with 1.5% chitin-glucan | 94 |

Effects of the UVB irradiation on the number of healthy Langerhans cells present in the explant. A first topical application is carried out, the explant is incubated for 24 hours, and then, one hour after a second topical application, the explants are irradiated with UVB radiation (0.75 J/cm²) in the UVB+ group, and not irradiated in the UVB-group. The explants are then incubated for 16 hours. Two explants per group are used.
Immunohistochemisty—The explants are frozen, and three sections per explant are fixed in an acetone/methanol mixture and then incubated with an anti-CD1a-FITC antibody (AbCys LO-CD1a-F05) and the Hoechst nuclear stain for 1 hour. The sections are observed by fluorescence microscopy. Only the Langerhans cells having a marked fluorescence and a “normal” morphology demonstrated by the presence of dendrites are counted.
A degree of protection compared with the non-UVB-irradiated control is calculated according to the following formula:

$$P ( \% ) = \frac{{LCtreated}_{UV +} - {LCcontrol}_{UV +}}{{LCcontrol}_{UV -} - {LCcontrol}_{UV +}} \times 100$$

### Where:

−LCtreated_{UV+} is the number of Langerhans cells in the treated explant exposed to UVB radiation.
LCcontrol_{UV+} is the number of Langerhans cells in the untreated explant exposed to UVB radiation.
LCcontrol_{UV-} is the number of Langerhans cells in the untreated explant not exposed to UVB radiation.
   The results are those in table D4.2.

TABLE D4.2
Number of Langerhans cells (LC) labeled with an anti-CD1a antibody
in the epidermis of a skin explant treated with a chitin-glucan-based
cream and exposed (UVB+) or not exposed (UVB−) to UVB
radiation, in comparison with an untreated control and a reference
treated with a factor-20 antisun cream; degree of protection

| | Healthy LC | Healthy LC | |
|---|---|---|---|
| | (number/mm²) | (number/mm²) | |
| Topical treatment | UVB− | UVB+ | P (%) |
| Control | 194.3 ± 53.7 | 35.2 ± 34.7 | 0 |
| 1.5% chitin-glucan cream | 233.3 ± 69.6 | 116.4 ± 57.7 | 51 |
| SPF20 sun cream | 187.4 ± 58.1 | 230.9 ± 68.4 | 100 |
| (reference) | | | |

It is concluded from this example that the topical application of a cream based on chitin-glucan with a particle size of less than 125 μm at the concentration of 1.5% makes it possible to maintain a large part of the Langerhans cells in the epidermis of a skin explant having undergone exposure to UVB radiation. A degree of protection of 51% against damage related to UVB exposure is calculated, a factor-20 antisun cream having a degree of protection of 100%. This protective effect against UVB radiation contributes to the anti-aging effect of the chitin-glucan compound.

### Example ‘E’

### Effects of the Oral Administration of a Chitin-Glucan Powder with a Article Size of Less Than 500 μm on the Parameters Characterizing the Cardiovascular Risks in Humans

This example aims to demonstrate the anti-atherosclerosis, antioxidant, blood-cholesterol-lowering and blood-lipid-lowering effect of the oral administration of chitin-glucan with a particle size of less than 500 μm in humans The model used is a human being who has a normal weight or is slightly overweight and who has a cholesterolemia of between 1.3 and 2.5 g/l on a standard diet. The chitin-glucan with a particle size of less than 500 μm (obtained according to the method of example A5) is administered at a rate of 4.5 g/day, as 3 intakes, 30 minutes before the main meals. The effects over a period of 4 weeks are studied. The control group receives the equivalent of 4.5 g/day of placebo. Said placebo is pharmaceutical-grade heavy kaolin for internal use. 30 male individuals 20 to 50 years old, having a body mass index of between 18 and 28 kg/m² and a cholesterolemia of between 1.3 and 2.5 g/l are divided up into two groups randomly and single-blind (10 controls/20 treated). A medical consultation and tests on the blood parameters considered for the study are carried out and a food questionnaire is filled out during the selection of the individuals. The food consumption, the biochemical and antioxidant parameters and the anthropometric parameters are measured 3 times over the course of the study (at the beginning, at 2 weeks and at the end of the study). The blood samples make it possible to analyze the biochemical and antioxidant parameters reiterated in table E1. The anthropometric measurements relate to the monitoring of the weight, the height measurement, the waist measurement, the arm circumference, the thigh circumference and the arterial pressure.

Regular consumption of plant polysaccharides in the form of a powder with a controlled particle size, such as the chitin-glucan, exerts a preventive effect on metabolic diseases such as hypercholesterolemia, cardiovascular diseases or, by extension, metabolic syndrome and obesity.

TABLE E1
Biochemical and antioxidant parameters monitored over
the course of the experiment in humans

| | | Halfway | |
|---|---|---|---|
| Selection of | Beginning of the | through the | End of the study |
| individuals | study (day 1) | study (day 15) | (day 29) |
| Hematology: | | | |
| Hemoglobin | | | |
| Hematocrit | | | |
| Complete | | | |
| blood | | | |
| Platelets | | | |
| Biochemistry: | Biochemistry: | Biochemistry: | Biochemistry: |
| Glucose | Glucose | Glucose | Glucose |
| Urea | Urea | Urea | Urea |
| Creatinine | Creatinine | Creatinine | Creatinine |
| Uric acid | Uric acid | Uric acid | Uric acid |
| TGO | TGO | TGO | TGO |
| TGP | TGP | TGP | TGP |
| γ-GT | γ-GT | γ-GT | γ-GT |
| Insulin | Insulin | Insulin | Insulin |
| Triglycerides | Triglycerides | Triglycerides | Triglycerides |
| Total | Total cholesterol | Total cholesterol | Total cholesterol |
| cholesterol | | | |
| HDL- | HDL-cholesterol | HDL-cholesterol | HDL-cholesterol |
| cholesterol | | | |
| | Oxidative | Oxidative | Oxidative |
| | assessment: | assessment: | assessment: |
| | Isoprostanes | Isoprostanes | Isoprostanes |
| | Lipid peroxides | Lipid peroxides | Lipid peroxides |
| | Oxidized LDLs | Oxidized LDLs | Oxidized LDLs |
| | Oxidized proteins | Oxidized proteins | Oxidized proteins |
| | Reduced | Reduced | Reduced |
| | glutathione | glutathione | glutathione |
| | Oxidized | Oxidized | Oxidized |
| | glutathione | glutathione | glutathione |
| | Vitamin C | Vitamin C | Vitamin C |
| | β-carotene | β-carotene | β-carotene |
| | α- and γ- | α- and γ- | α- and γ- |
| | tocopherols | tocopherols | tocopherols |

TABLE E2
Influence of taking chitin-glucan on biochemical and
antioxidant parameters

| | Influence of taking |
|---|---|
| | chitin-glucan |
| Total cholesterol | ≅ |
| Triglycerides | [symbol] |
| HDL-cholesterol | [symbol] |
| LDL-cholesterol | [symbol] |
| Antioxidizing capacity | [symbol] |
| Uric acid | [symbol] |
| Creatinine | [symbol] |
| Urea | [symbol] |

It is noted that taking chitin-glucan with a particle size of less than 500 μm, orally, significantly improves the lipid and antioxidant profiles and the associated parameters in human individuals. This makes it possible to conclude that the regular consumption of chitin-glucan is beneficial in the prevention of atherosclerosis and, by extension, with respect to the associated pathologies.

### Series of Examples ‘F’

### Porous Cohesive Materials Comprising at Least Chitin-Glucan with a Fine and Controlled Particle Size

### Example F1

### Use of the Chitin-Glucan in the Form of a Porous Material

A chitin-glucan paste is prepared by homogenizing 100 g of a chitin-glucan powder with a fine particle size (L25, fraction <90 μm) with 900 g of water, for at least 1 hour. The paste is frozen at −18° C., and then lyophilized. A cohesive porous material with good mechanical strength is obtained. Observation by scanning electron microscopy (FIG. 6) reveals a very aerated, isotropic and fibrillar structure.

### Example F2

### Use of the Chitin-Glucan and of Chitosan in the Form of a Cohesive Porous Composite Material

A solution of chitosan at 2% in 1% acetic acid is prepared. A chitin-glucan powder with a fine particle size (L16, fraction <90 μm) is dispersed therein and the dispersion is homogenized for 2 minutes. The dispersion is frozen at −18° C. and then lyophilized. A cohesive porous material with good mechanical strength is obtained. Observation by scanning electron microscopy (FIG. 7, longitudinal section) reveals a nonfibrillar, interconnected porous structure, the pores exhibiting a certain orientation.

### Example F3

### Preparation of a Porous Composite Material Having Chitosan as Polymer Matrix and Particles of Chitin-Glucan Copolymer as Dispersed Agent: Influence of the Particle Size on the Mechanical and Morphological Properties

Various chitin-glucan particle sizes are prepared according to the method of example A5. The fraction having a particle diameter of less than 63 μm, and the fractions having a diameter of between 125 and 250 μm, 250 and 500 μm, and 500 and 1000 μm, are separated by screening.

Chitosan having a molecular mass of 42 K (molecular mass determined by capillary viscometry) and a degree of acetylation of 11% is dissolved in acetic acid (1%) so as to form a solution with a concentration equal to 2% (m/m). A given weight of particles of chitin-glucan with a controlled particle size is added to a given volume of this solution. A volume of 4 ml of suspension is homogenized by magnetic stirring for 2-3 min before being poured into a mold (diamond-shaped) and frozen. The sample is then placed on the shelf of a lyophilizer in order to eliminate the solvent by sublimation under vacuum for 48 h.
Cohesive porous composite materials having various compositions were prepared by varying the weight proportion of chitosan and of chitin-glucan and also the particle size of the chitin-glucan powder.
Composite materials in the form of foams of various formats were produced by varying the size of the mold—examples: hexagonal polystyrene weighing boats, small format: 4 ml of solution; large format: 15 ml of solution. The parameters for formulation of the foams prepared are reiterated in table F3.1 below.

TABLE F3.1
Chitosan/chitin-glucan mixtures and properties of the cohesive porous
composite materials

| | Proportion | Chitin- | | | Young's |
|---|---|---|---|---|---|
| | chitosan/chitin | glucan | | Density | modulus |
| Ref | glucan (m/m) | fraction | | (g/cm³) | (MPa) |
| A2 | 25/75 | <63 | μm | 0.104 | 0.16 ± 0.02 |
| B2 | 50/50 | <63 | μm | 0.052 | 0.47 ± 0.15 |
| C2 | 75/25 | <63 | μm | 0.038 | 0.14 ± 0.09 |
| A3 | 25/75 | 125-250 | μm | 0.114 | 0.23 ± 0.01 |
| B3 | 50/50 | 125-250 | μm | 0.060 | 0.07 ± 0.01 |
| C3 | 75/25 | 125-250 | μm | 0.037 | 0.15 ± 0.05 |
| A5 | 25/75 | 500-1000 | μm | 0.114 | 0.06 ± 0.02 |
| B5 | 50/50 | 500-1000 | μm | 0.060 | 0.25 ± 0.06 |
| C5 | 75/25 | 500-1000 | μm | 0.039 | 0.31 ± 0.01 |

The composite materials in the form of foams obtained by lyophilization were characterized in terms of microstructure by scanning electron microscopy (SEM). The compression strength of the foams, expressed by the Young's modulus, is determined by means of axial compression tests on an Instron 5566 tensile-compression testing machine, equipped with a low force cell. The samples were subjected to a preload of 0.03 N, and deformed with a speed of 0.2 mm/min. The Young's modulus is determined from the initial linear region of the stress/strain curve. The density was determined by gravimetric analysis (volume/mass of the foam). The results are reported in table F3.1. The SEM micrographs are those of FIG. 8.
It is thus understood from table F3.1 that the density of the foams increases as the proportion of the chitin-glucan increases. Surprisingly, it is noted that the Young's modulus is the highest for an equivalent chitosan/chitin-glucan proportion (50/50), whereas it is significantly lower for the proportions 75/25 and 25/75. A proportion of approximately 50% of chitin-glucan particles thus constitutes the optimal proportion for improving the mechanical properties of the foams and, in the case in point, the axial compression strength.
It is also noted on FIG. 8 that the chitin-glucan particles are distributed homogeneously over the thickness of the foam, their density increasing logically with their initial proportion. The particles are found here and there within the pores and often anchored in the very walls of the pores.
Recordings of images obtained by SEM (FIG. 9) carried out for samples A5 and B5 (size of the chitin-glucan particles between 500 and 1000 μm) and also for samples A4 and B4 (size of the particles between 250 and 500 μm) reveal that the structural homogeneity no longer exists. The particles are too large in size and sediment and concentrate in the lower part of the sample after lyophilization. This example demonstrates that, with a view to using the chitin-glucan copolymer in a porous composite material, this copolymer should have a fine and controlled particle size.

1. A fungal extract, wherein said fungal extract comprises micrometric particles of at least one chitin-glucan copolymer, or a hydrolysate thereof, and wherein at least 70% by weight of said micrometric particles have a size less than 500 microns (μm).

2. The fungal extract of claim 1, wherein at least 70% by weight of said micrometric particles of have a size less than 355 microns (μm).

3. (canceled)

4. (canceled)

5. The fungal extract of claim 1, wherein said fungal extract is obtained from a fungus chosen from the group made up of a fungus of the Ascomycete type, of Aspergillus niger type, of Basidiomycete type, of Lentinula edodes (shiitake) type and of Agaricus bisporus (button mushroom) type, and any mixture thereof.

6. (canceled)

7. The fungal extract of claim 1, wherein the micrometric particles are made up of at least 50% by weight of particles with a size of less than 250 microns (μm).

8. The fungal extract of claim 1, wherein the micrometric particles have a size of less than 125 μm.

9. The fungal extract of claim 1, wherein the micrometric particles have a size of less than 90 microns (μm).

10. A composition comprising a fungal extract as defined in any one of claims 1 to 9.

11. The composition as claimed in claim 10, wherein the composition is a cosmetic composition.

12. The composition as claimed in claim 10, wherein said fungal extract is used at a concentration of between 0.01% and 10% by weight of the total composition.

13. The composition as claimed in claim 10, wherein said composition is in the form of a suspension, of an emulsion or of a dispersion comprising a solvent and at least one said fungal extract.

14. A method for practicing a care selected from a cosmetic care, a dermocosmetic care, a dermatological care, a care is selected from the group consisting of a body care, a face care, a care for improving the hydration of the skin, a care for increasing the capacity of the skin to retain water, a care for improving the barrier function of the skin, a care for improving the protection of the skin and/or the defense activities of the skin, a care for exerting an anti-aging effect, a care for decreasing wrinkles or slowing down or preventing the appearance of wrinkles, a care for improving the appearance of the skin, a care for improving the homogeneity of the skin, a care for improving the firmness and the tonicity of the skin, a care for promoting the attachment of the epidermis to the dermis, and a pharmaceutical care, said pharmaceutical care being selected from the group consisting of a care for obtaining an effect chosen from the group consisting of an antioxidant, blood-cholesterol-lowering or blood-lipid-lowering effect, a stimulators effect on the immune system, a hypoglycemic effect, and an effect consisting in preventing and/or combating a pathology selected from the group consisting of dyslipidemia, atherosclerosis, obesity, an obesity-related disease, a cardiovascular disease, metabolic syndrome, diabetes and hyperuricémie, said method comprises using a composition as defined in any one of claims 1 to 9.

15. (canceled)

16. (canceled)

17. A food supplement composition comprising, as active ingredient, at least one extract of fungal origin, as defined in any one of claims 1 to 9.

18. A pharmaceutical composition comprising, as active ingredient, at least one extract of fungal origin, as defined in any one of claims 1 to 9.

19. (canceled)

20. A method for preparing a fungal extract comprising micrometric particles of at least one chitin-glucan copolymer, or a hydrolysate thereof, wherein said method comprises:
a) the extraction and purification of a chitin-glucan copolymer from a fungal biomass, said copolymer of this fungal extract being insoluble in water or an organic solvent,
b) steps, which are simultaneous or separate independently of their order, of filtration, drying, milling and classification of the particles obtained after milling, starting from dry or solvated chitin-glucan, making it possible to obtain micrometric particles in which at least 70% by weight of the particles have a size of less than 500 microns (μm).

21. A porous material comprising at least one fungal extract comprising micrometric particles of at least one polymer selected from the group consisting of a chitin polymer, a chitin-glucan copolymer, a hydrolysate thereof, and a combination thereof, said micrometric particles having a particle size of less than 500 microns (μm).

22. (canceled)

23. The porous material as claimed in claim 21 wherein the particle size is less than 250 microns.

24. A method for preparing a porous material, wherein said method comprises a step of dispersing or of emulsifying or of suspending in a solvent at least one fungal extract in the form of particles with a particle size of less than 500 microns, then the elimination of this solvent, and the obtaining of a porous material comprising the fungal extract.

25. A porous composite material comprising a matrix and a dispersed agent, said matrix, also known as dispersing agent, being at least one type of polymer, and the dispersed agent being at least one fungal extract in the form of particles with a particle size of less than 500 microns (μm).

26. (canceled)

27. The porous composite material as claimed in claim 25, wherein the particle size is less than 250 microns.

28. A method for preparing a porous composite material comprising a matrix and an agent dispersed in the matrix, wherein said method comprises (i) solubilizing a polymer capable of forming the matrix of the porous composite material, (ii) dispersing, or emulsifying, or suspending at least one fungal extract in the form of particles with a particle size of less than 500 microns, in the solution of polymer, (iii) of eliminating the solvent from the solution of polymer comprising the fungal extract, (iv) the obtaining of a composite material comprising the porous polymer forming the matrix and the fungal extract forming the dispersed agent.

29. The fungal extract of claim 1, wherein the micrometric particles have a size comprised between 50 microns (μm) and 90 microns (μm).
